(19) United States
(12) Patent Application Publication
(10) Pub. No.: US 2026/0137443 A1
(43) Pub. Date: May 21, 2026

(54) REAL-TIME IMPEDANCE-BASED GUIDEWIRE NAVIGATION IN ENDOVASCULAR INTERVENTIONS

(71) Applicant: Lumen Medical, Inc., Santa Clara, CA (US)

(72) Inventors: Luke Peter Dawson, Durham, NC (US); Michael Harmer Haines, Melbourne (AU); David Angus Begg, Melbourne (AU)

(21) Appl. No.: 19/387,398

(22) Filed: Nov. 12, 2025

Related U.S. Application Data
(60) Provisional application No. 63/721,383, filed on Nov. 15, 2024.

(51) Int. Cl. A61B 18/14; A61B 18/00; A61B 18/12

(52) U.S. Cl. CPC A61B 18/1492; A61B 18/1206; A61B 2018/00351; A61B 2018/0072; A61B 2018/00875; A61B 2018/00892; A61B 2018/124

(57) ABSTRACT

Systems and techniques are disclosed for improving real-time tissue characterizations during endovascular procedures. In some implementations, a method involves characterizing biological tissue for an endoluminal procedure. A first electrode is positioned within an endoluminal space adjacent to a region of biological tissue. A second electrode is positioned spatially separated from the first electrode. A variable-frequency input electrical signal is applied across the first electrode and the second electrode to generate an electrical field corresponding to the region. A voltage response is measured between the first electrode and the second electrode based on the electric field. A voltage phasor is determined based on the voltage response relative to the variable-frequency input electrical signal. A characteristic of the region is determined based on the voltage phasor.

### CROSS-REFERENCE TO RELATED APPLICATIONS

This application claims the benefit of U.S. Provisional Application No. 63/721,383, filed Nov. 15, 2024, and titled “SYSTEM AND METHOD FOR REAL-TIME IMPEDANCE-BASED GUIDEWIRE NAVIGATION IN CHRONIC TOTAL OCCLUSION AND VASCULAR INTERVENTIONS,” the entire contents of which are incorporated by reference in their entirety.

### TECHNICAL FIELD

This disclosure relates generally to endovascular intervention devices, and more particularly to impedance-based guidewire devices.

### BACKGROUND

Endovascular interventions rely on the ability to precisely navigate devices such as guidewires and catheters through complex vascular pathways to reach target treatment sites. In some procedures, particularly those involving narrowed, tortuous, or obstructed vessels, clinicians may be limited by incomplete feedback about the physiological characteristics of the tissue surrounding the intravascular device. Imaging techniques such as fluoroscopy provide anatomical guidance but offer limited real-time information about the local tissue environment. This can complicate navigation, increase procedural time, and elevate the risk of complications. While intravascular imaging tools may offer enhanced visualization, they often involve additional hardware and larger delivery profiles that are not always compatible with the targeted anatomy and may result in their own complications. Accordingly, there is a need for systems and methods that can provide real-time physiological or electrical information during endovascular navigation.

As one example, during CTO interventions, operators often lack real-time feedback about the location and surrounding tissue characteristics of the guidewire as it advances through fully occluded vessels. Because contrast media cannot pass through the occlusion, traditional fluoroscopic imaging provides limited insight beyond the blockage. As a result, clinicians typically rely on tactile feedback and trial-and-error with multiple wires, which can extend procedure time, increase radiation exposure, and elevate the risk of complications such as vessel perforation. While some adjunctive tools like intravascular ultrasound (IVUS) exist, these often involve larger delivery systems that are difficult to use within the constrained anatomy of a CTO. Additionally, if the wire is not in the expected position (e.g., if it has perforated outside the vessel), insertion of adjunctive tools like IVUS may lead to complications by enlarging a perforation resulting in bleeding and sometimes pericardial effusion and/or death. Accordingly, there is a need for techniques that provide real-time physiological or electrical characterization of tissue in proximity to the guidewire tip without modifying the guidewire or altering the clinical workflow.

### SUMMARY

The systems and methods described herein may also be beneficial in a variety of endovascular procedures beyond coronary CTO interventions. For example, in peripheral artery disease (PAD) interventions, impedance-based tissue feedback may assist in navigating long-segment occlusions or identifying transitions between healthy vessel walls and calcified plaques. In neurovascular procedures (e.g., those involving the treatment of cerebral aneurysms or ischemic stroke), real-time physiological information supports safer guidewire advancement through delicate or tortuous vasculature. Similarly, in venous interventions like recanalization of chronically occluded central veins (e.g. from pacing wires) or treatment of post-thrombotic syndrome, electrical sensing may help distinguish occlusions from patent venous channels and identify wire perforations outside the vessel. In pulmonary arterial procedures, such as balloon pulmonary angioplasty, electrical sensing may help distinguish organized chronic thromboembolic occlusions and avoid perforations. In interventional radiology procedures, guidewire sensing may improve safety and accuracy in transiting through different tissues, for example in transjugular intrahepatic portosystemic shunt (TIPS) procedures, where a guidewire is passed from the portal vein through a portion of the liver and into the hepatic vein. Even in structural heart procedures, such as transseptal puncture, Bioprosthetic or native Aortic Scallop Intentional Laceration to prevent latrogenic Coronary Artery obstruction (BASILICA), Laceration of the Anterior Mitral leaflet to Prevent Outflow Obstruction (LAMPOON), or Septal Scoring Along Midline Endocardium (SESAME) procedures, localized tissue characterization may provide operators with additional feedback to improve precision and reduce risk. In these contexts, the ability to identify tissue types in real-time by obtaining real-time impedance-based measurements (apparent impedance) without significantly altering procedural workflows offers practical and versatile advantages.

To address the need for real-time physiological feedback during guidewire advancement in occluded vessels, this disclosure presents concepts that advance the field of endovascular interventions by enabling real-time characterization of tissue near the guidewire tip through the use of catheter systems configured with multiple guidewires. These systems facilitate impedance-based tissue analysis with or without requiring modification to conventional guidewires or reliance on intravascular imaging modalities. In some implementations, the guidewires are positioned in different coronary vessels adjacent to an occluded region, allowing one or more electrical signals to be applied, creating a varying electrical field across the tissue of interest. By measuring the resultant electrical potential at the tip of the guidewire, the surrounding tissue types can be characterized, providing the operator with real-time physiological information to support decision-making during procedures such as CTO interventions.

Implementations described within this disclosure provide improvements to existing guidewire navigation techniques for endovascular interventions. Some implementations enable real-time tissue characterization during coronary vascular procedures by acquiring impedance-based measurements directly through guidewires positioned within the vasculature. In contrast to some imaging-based approaches (e.g., intravascular ultrasound (IVUS), optical coherence tomography (OCT)), the systems described herein utilize the guidewires themselves as electrodes, eliminating the need for dedicated imaging catheters. This approach reduces device profile, allowing clinicians to operate within the narrow and tortuous anatomy typical of chronic total occlusions (CTOs) without additional hardware or workflow disruptions. Moreover, because the measurements are acquired as the wire advances, the system can provide continuous physiological feedback that augments fluoroscopic imaging and improves procedural awareness.

As described herein, various electrode configurations are contemplated to accommodate differing clinical needs and anatomical constraints. For example, in a bipolar electrode configuration, two electrodes (e.g., positioned on separate wires) are used to both deliver current and measure voltage, enabling straightforward data acquisition with minimal hardware. As another example, in a tetrapolar electrode configuration, one pair of electrodes delivers current and a second, separate pair senses the resulting voltage. The tetrapolar electrode configuration helps eliminate measurement artifacts caused by electrode polarization and capacitive coupling, which are known to degrade signal fidelity in low-frequency biological impedance systems. The tetrapolar approach offers improved measurement precision and tissue discrimination accuracy, even when using unmodified guidewires and commercially available catheters.

The disclosed systems also support advanced signal processing techniques with hardware flexibility. Examples of such signal processing techniques include Nyquist analysis, Cole-Cole modeling, and principal component analysis (PCA), among others. These techniques help extract tissue-specific electrical signatures from the data and thereby allow the system to distinguish between blood, fat, vessel wall, and myocardial tissue. Tissue distinctions may be achieved with high specificity, even in the presence of noise or partial wire insulation. In some implementations, the results of this analysis are rendered graphically to the operator in real time, offering a new layer of physiological context during guidewire manipulation. By combining familiar tools (e.g., guidewires, catheters) with non-invasive electrical sensing and modern data analytics, the disclosed systems enhance operator decision-making while preserving procedural simplicity and minimizing additional equipment burden.

In one general aspect, a method is disclosed for characterizing biological tissue for an endoluminal procedure. The method includes positioning a first electrode within an endoluminal space adjacent to a region of biological tissue. The method also includes positioning a second electrode spatially separated from the first electrode. Further, a variable-frequency input electrical signal is applied across the first electrode and the second electrode to generate an electrical field corresponding to the region. A voltage response between the first electrode and the second electrode based on the electric field is measured. A voltage phasor is determined based on the voltage response relative to the variable-frequency input electrical signal. The method also includes determining a characteristic of the region based on the voltage phasor.

One or more implementations may include the following optional features. For example, in some implementations, the method also includes positioning a third electrode and a fourth electrode that are each spatially separated from the first electrode. In such implementations, the variable-frequency input electrical signal is applied across the third electrode and the fourth electrode. Additionally, the voltage response is measured between the first electrode and the second electrode.

In some implementations, the first electrode is carried by an elongated member. Additionally, the elongated member includes a guidewire, a catheter, or a sheath.

In some implementations, the elongated member includes an insulating sheath. Additionally, the method further includes retracting the insulating sheath relative to a body of the elongated member to adjust a length of an exposed region of the first electrode.

In some implementations, the region of biological tissue includes a body vessel.

In some implementations, positioning the second electrode includes positioning the second electrode on an external skin surface of a subject.

In some implementations, the variable-frequency input electrical signal includes a frequency sweep across a test frequency band.

In some implementations, the test frequency band is within a range of 1 kilohertz to 1 gigahertz. Additionally, in such implementations, the input electrical signal has a current amplitude within a range of 0.1 microamperes to 10 milliamperes.

In some implementations, determining the characteristic of the region includes comparing the determined voltage phasor to a reference dataset using a machine learning model.

In some implementations, the method further includes displaying, on a user interface, a graphical representation of the characteristic.

In some implementations, the biological tissue includes cardiovascular, neurological, hepatic, renal, or musculoskeletal structures.

In some implementations, the characteristic identifies the region of biological tissue as one or more materials selected from a group including blood, subintimal layer, vessel wall, plaque, myocardium, pericardial space, fat, muscle, and clot.

In some implementations, the method further includes navigating the first electrode through a vascular pathway based on the characteristic.

In another general aspect, disclosed is a system for characterizing biological tissue for an endoluminal procedure. The system includes an elongated member having a first electrode. The first electrode is configured for positioning within an endoluminal space adjacent to a region of the biological tissue. The system also includes a second electrode configured for positioning to be spatially separated from the first electrode. Additionally, a signal generator is operatively coupled to each of the first electrode and the second electrode. The signal generator is configured to provide a variable-frequency input electrical signal across the first electrode and the second electrode to generate an electric field through the region. Further, a measurement unit is operatively coupled to each of the first electrode and the second electrode. The measurement unit is configured to measure a voltage response between the first electrode and the second electrode based on the electric field. The system also includes a processor operatively coupled to each of the measurement unit and the signal generator. The processor is configured to receive data indicating the voltage response. The processor is also configured to determine a voltage phasor based on the voltage response relative to the variable-frequency input electrical signal, and generate output data including a characteristic of the region based on the voltage phasor.

One or more implementations may include the following optional features. For example, in some implementations, the elongated member includes a proximal electrical interface. In such implementations, the first electrode is electrically coupled to the proximal electrical interface via a conductor disposed in the elongated member.

In some implementations, the system further includes a user interface configured to receive the output data from the processor and provide a visual representation of the characteristic.

In some implementations, the first electrode comprises a conductive surface on the elongated member that is electrically isolated from a body of the elongated member by an insulating material.

In some implementations, the processor is further configured to generate a control signal that, when received by the signal generator, causes the signal generator to adjust one or more of an excitation frequency or a signal amplitude of the variable-frequency input electrical signal based on the voltage response.

In some implementations, the processor is configured to generate the output data by executing a machine learning model to compare the voltage phasor to a reference dataset.

In some implementations, the system further includes a third electrode and a fourth electrode. In such implementations, the signal generator is operatively coupled to the third electrode and the fourth electrode and configured to apply the variable-frequency input electric signal across the third electrode and the fourth electrode.

### BRIEF DESCRIPTION OF DRAWINGS

FIG. 1 is a schematic representation of an exemplary system for generating an electrical test field and electrical measurement using guidewires as the electrodes.

FIG. 2A-2G are schematic representations of a structure of an example guidewire without and with modification.

FIG. 3A-3C are schematic representations illustrating examples of bipolar, tripolar, and tetrapolar electrode configurations in a tissue.

FIG. 3D is a schematic representation of an exemplary tetrapolar system.

FIGS. 4A, 4B, 4C, 4D, 4E, 4F, 4G, 4H, and 4I are schematic representations illustrating examples of common reference (R) and working sense (WS) electrode locations within a coronary vessel.

FIGS. 5A and 5B are schematic representations of examples of tetrapolar configurations in a coronary CTO intervention and peripheral CTO intervention, respectively.

FIGS. 6A and 6B are schematic representations of example skin electrode placements for a coronary CTO intervention and a peripheral CTO intervention, respectively.

FIGS. 7A and 7B are schematic representations illustrating current density based on the placement of W and C in a torso region.

FIGS. 8A, 8B, 8C, 8D, and 8E are schematic representations illustrating the effect of electrode placement on impedance measurements.

FIGS. 9A and 9B illustrate 3D representations of the coronaries to show an example of a guidewire position in a vessel.

FIGS. 10A and 10B illustrate an example of a guidewire position during a peripheral vascular procedure.

FIG. 11A is a schematic representation illustrating the anatomical positions of the subject's skin and cardiac system for a 4-electrode placement for EIS measurements.

FIG. 11B illustrates an exemplary position of the 4-electrode system in a porcine.

### DETAILED DESCRIPTION

The present disclosure relates to systems and methods for performing tissue characterization during endovascular and interventional procedures using electrical impedance-based measurements acquired through electrode carrying members, such as guidewires or catheters, positioned within the vasculature and/or other body lumens or cavities. In various implementations, a user may navigate one or multiple guidewires independently into separate vessels (e.g., coronary) to be adjacent to a tissue of interest (e.g., vessel intima, plaque, thrombosis, adipose or muscle tissue).

Electrical signals may be applied between two or more electrodes and the resultant electrical signal (i.e., a voltage phasor) is collected across the tissue of interest and compared to the input signal, with the deviation in signal phase and amplitude used to characterize the local tissue environment. In some implementations (e.g., bipolar configurations), the data may be a direct measurement of impedance across the area of interest just like electrochemical impedance spectroscopy (EIS). However, in other configurations (e.g., tetrapolar configurations with a distant reference), where electrode positioning makes a direct measure of impedance invalid, the relative phase and amplitude shift of the voltage signal are used as a surrogate variable. These measurements may provide real-time physiological feedback during the procedure, assisting the operator in assessing the guidewire's position relative to the surrounding anatomy and occlusion if present.

The techniques disclosed herein enable assessments with any suitable conductive guidewire, or customized electrode carrying member, avoiding the use of intravascular imaging systems, which may be particularly useful in complex endovascular and/or interventional procedures, for example in coronary chronic total occlusion procedures or other vascular territories.

As described herein, “guidewire” generally refers to an elongated, flexible medical device configured for advancement through a vascular structure to facilitate the placement, navigation, or operation of other endovascular tools or catheters. A guidewire may include one or more conductive regions along its axial length and may be partially or fully coated with insulating material depending on its intended use. The guidewire may be commercially available or custom-fabricated and may be used in its standard clinical form or integrated with additional sensing or signal transmission elements. In the context of various implementations, the guidewire may also function as an electrical conductor for delivering or sensing signals and may be positioned in an endoluminal position either alone or in combination with other guidewires or catheter-based systems. Unless otherwise specified, the term is intended to encompass a range of designs, materials, and configurations consistent with clinical practice in coronary and peripheral endovascular interventions.

As described herein, “catheter” generally refers to an elongate, flexible medical device configured for insertion into a body lumen, such as a blood vessel, for diagnostic or therapeutic purposes. A catheter may include one or more lumens, structural reinforcements, or conductive elements, and may be used to support or deliver guidewires, fluids, electrodes, sensors, or other tools. In the context of the disclosed implementations, the catheter may also provide a structural platform for positioning multiple guidewires within a vascular region or for facilitating electrical connections used in impedance-based tissue characterization. Unless otherwise specified, the term is intended to encompass a range of commercially available and custom-designed devices, including sheaths, delivery systems, and composite devices used in endovascular procedures.

As described herein, “endoluminal intervention” generally refers to any medical procedure involving the insertion and manipulation of devices within internal lumens within a patient's body (e.g., blood vessel) for diagnostic or therapeutic purposes. Such interventions may be performed in coronary, peripheral, cerebral, or other vascular territories, and typically involve the use of catheters, guidewires, or other interventional instruments to access or treat endoluminal structures. Endoluminal interventions include, but are not limited to, procedures for revascularization, embolization, thrombectomy, or diagnostic evaluation. One example of an endoluminal intervention is an endovascular CTO procedure, in which guidewires are advanced through a completely occluded vascular segment (often in a coronary artery) with the goal of restoring perfusion. The systems and methods described in this disclosure may be applicable to CTO procedures as well as a variety of other endoluminal interventions where real-time physiological or electrical information about vessel-adjacent tissue may assist in clinical decision-making.

As described herein, “vascular structure” generally refers to any anatomical structure within the circulatory system that conveys blood, including arteries, veins, capillaries, or vascular grafts. The term is intended to encompass both natural and artificial conduits, and includes structures found in coronary, peripheral, cerebral, and visceral circulations. In the context of the disclosed systems and methods, a vascular structure may also refer to an occluded or partially occluded segment of a vessel in which diagnostic or therapeutic procedures (e.g., guidewire navigation or impedance sensing) are performed.

As described herein, “real-time” generally refers to the acquisition, processing, and presentation of data with minimal delay such that the information is available to the operator during the course of a medical procedure without interrupting or postponing the clinical workflow. For example, impedance-based measurements acquired through a guidewire may be processed and displayed while the guidewire is being advanced through a vascular structure. In certain instances, data may be collected in real-time without instantaneous processing but with a delay that is less than, for example, processing delays associated with post-procedural or offline data analysis.

As described herein, “voltage phasor” generally refers to a frequency domain representation of the time-varying voltage signal measured between the WS and R electrodes. The voltage phasor is characterized by a magnitude and a phase angle, which together define the sinusoidal component of the voltage signal. The phase angle at each frequency step is expressed relative to the phase of the corresponding input current signal. Thereby enabling determination of impedance or other phase-dependent electrical properties.

Some endovascular procedures (e.g., CTO intervention in a coronary vessel or peripheral vascular interventions) involve several technical challenges. These challenges include the inability to visualize the true vessel path beyond the occlusion, uncertainty about whether the guidewire remains within the true vessel lumen, the vessel wall, or has exited the vessel entirely, and an increased risk of vessel perforation due to uncertainty in guidewire positioning. Additional challenges include confirming successful passage through the occlusion and the need for stiffer guidewires to penetrate the hardened plaque.

To address these and other challenges, disclosed implementations provide systems and methods for improved ways of monitoring the position of a guidewire within a tissue during an endovascular intervention. In some instances, this may also be useful for tissue characterizations (e.g., characterizing a tissue type based on electrical impedance measurements obtained through the guidewires). The guidewires used in the endovascular procedures may serve as an electrode for the electrical impedance measurements.

The systems may include a signal generator, one or more electrode carrying elongated members (such as guidewires), other electrodes (e.g., surface electrodes), a measurement unit, a processor running a software application, and a display unit. The system can be set up in a bipolar, tripolar, or tetrapolar configuration using various configurations of internal electrode(s), internal or external reference electrode(s), and/or external skin patch electrode(s). The ability of electrodes to both inject and record electrical signals when in contact with the body is facilitated by the inherently conductive nature of body tissues, allowing electrical interactions. For example, tissues such as blood, fat, myocardium, muscle, skin (wet), skin (dry), plaque, and subintima have conductivity values of about 0.7008, 0.024, 0.195, 0.352, 0.029, 0.00027, 0.025, and 0.065 siemens/meter (S/m), respectively. These same tissues exhibit relative permittivity values of 5197.7, 172.42, 16982, 10094, 21876, 1126.8, 100, and 650, respectively.

Measurement configurations involve a current field injection electrode pair and a voltage sensing electrode pair. The current field injection pair consists of two electrodes referred to as the working (W) and counter (C) electrodes. These pass a variable current between them creating a measurable electrical field in the region of interest. The voltage sensing pair consists of a working sense (WS) and a reference (R) electrode. These are used to measure the potential between two points within the generated field.

Bipolar configuration measurements are performed using only two electrodes. The first electrode functions as both the working and working sense (W/WS) and the second functions as both the counter and reference (C/R). The W/WS electrode will be positioned within the tissue of interest and will be an electrode on an elongated member (e.g., commercial or custom guidewire). While the C/R electrode will be placed in other tissue and may be either on an elongated member or an external electrode (e.g., an adhesive skin electrode). In some implementations, both the W/WS electrode and C/R electrode can be placed on the same elongated member. In such cases, the elongated member can be modified with dual insulated conductive cores.

In a tripolar configuration three electrodes are used, one functions as the W, another as the WS, and the third as the C/R. The WS is placed in the tissue of interest and will be an electrode on an elongated member (e.g., guidewire). While the W and C/R are positioned to generate the current field through the area. The W and C/R electrodes may be placed internal or external in contact with other tissues (e.g., blood, skin), and both can include a variety of different electrode types, including an electrode on an elongated member, a skin patch electrode, or other external electrodes. In another implementation, a baseline measurement is taken while the WS electrode is not in the tissue of interest (e.g., while in blood). When a measurement is then taken with the WS in the tissue of interest, it is then re-referenced to the baseline measurement.

In a tetrapolar configuration four separate electrodes are used, each functioning as either the W, WS, R, or C. Like in the tripolar configuration, the WS is placed in the tissue of interest and will be an electrode on an elongated member. Also, the W and C are positioned to generate the current field through the area and can include internal or external electrodes. The R electrode can be placed internally or externally and be either an electrode on an elongated member or a surface electrode.

In some implementations, external electrodes (e.g., silver/silver chloride (Ag/AgCl) adhesive skin patch electrodes) are used as the current carrying electrodes (i.e., W and C), while the guidewires placed in the vasculature function as the WS and R. In some cases, a single guidewire can be modified to include multiple electrodes, including a combination of the current injector (i.e., W and C) and voltage sensing (i.e., WS and R) electrodes.

The signal generator is used to apply a low amplitude alternating current (AC) through the working and counter electrodes, and the voltage difference is measured across the working sense and reference electrodes. In some cases, the voltage measured in the tissue of interest can be re-referenced to a baseline measurement. The baseline measurement is determined when the sensing electrode is initially placed in blood. The difference in voltage can be measured between the different sweeps. The measured voltage difference is used to calculate complex impedance or the voltage phasor as a surrogate that includes real and imaginary components. The resulting data can be visualized on a Nyquist plot, Cole-Cole plot, or Bode plot, which can be used to characterize the type of tissue encountered during the endovascular procedure, for example, distinguishing between blood, vessel wall, plaque, and other tissue types. In some implementations, algorithms that are processed by a computer are used to classify the tissue type based on the data.

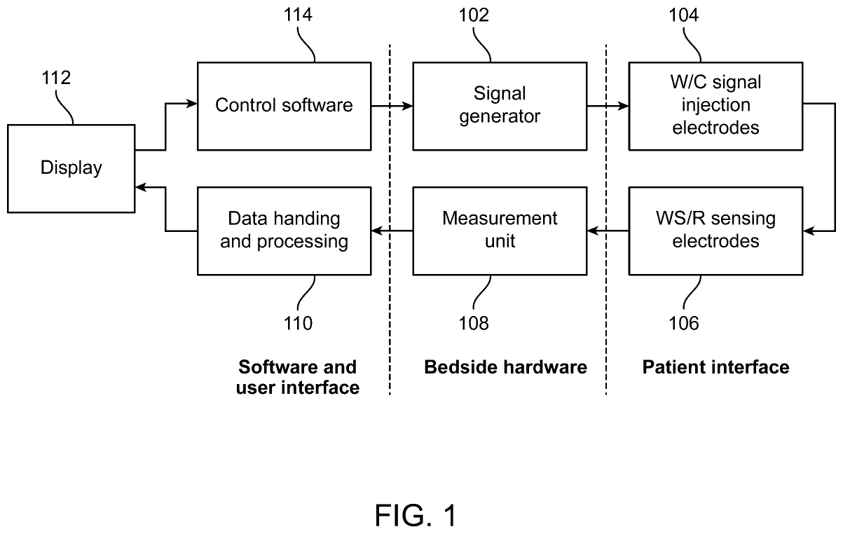

FIG. 1 is a schematic representation of an exemplary system for generating an electrical test field and electrical impedance or voltage phasor measurement using guidewires as the electrodes. The system enables real-time tissue characterization during endovascular interventions. The endovascular interventions can include, but not limited to, coronary CTO, peripheral CTO, interventional neurology, interventional nephrology, interventional oncology, interventional radiology, among others. The system includes a signal generator 102, which supplies a low amplitude AC current to the working and counter signal injection electrodes 104 at a single frequency. The peak amplitude of the low AC current can range between 1 μA-1 mA, if the current injecting electrodes are intravascular, and from 10 μA-10 mA if the current injecting electrodes are on the skin. The frequency can range between 1 kHz-1 MHz when standard electrical impedance spectroscopy (EIS) is being used (where current is applied into the tissue of interest). The frequency can range from 1 kHz to 100 MHz when a current field is applied to the body to allow measurement of voltage amplitude and phase to determine the dielectric response characteristics of different tissues (i.e., modified dielectric spectroscopy using distributed current fields), which is within the safety limits for a medical electrical equipment. In some implementations, a multi-frequency electrical signal is applied to the current carrying electrodes.

The current carrying electrodes 104 can include a single guidewire or multiple guidewires. In some implementations, external skin patches (e.g., Ag/AgCl adhesive patches, 50×50 mm, or 50×70 mm) are placed on the subject's skin and used as the current carrying electrodes 104. The Ag/AgCl skin patches are biocompatible adhesives. In some implementations, two external skin patches are positioned on the surface of the skin as the W and C electrodes.

The sensing electrodes 106 can include one or more guidewires placed in the vasculature. In certain implementations, two guidewires are placed intravascularly and function as the voltage sensing electrodes (WS and R). The intravascular guidewires can include unmodified coronary or peripheral guidewires with an exposed tip. The intravascular guidewires have a conductive core made of stainless steel or nitinol with an insulated shaft, connected via a custom spindle clip to allow free wire rotation by the operator. In some implementations, the sensing electrode R can include an external skin patch (e.g., Ag/AgCl) or a custom catheter. When an electric field is generated by the current-carrying electrodes 104, the voltage difference across the sensing electrodes 106 is measured to enable assessment of tissue dielectric response characteristics. Voltage measurements in this setup use high input impedance so that minimal current flows through the guidewire in the tissue of interest (improving safety when the guidewire is intracardiac).

The measurement unit 108 is used to measure the voltage difference across the voltage sensing electrodes WS and R. The measurement unit 108 can include a commercial device, such as a potentiostat, which incorporates circuitry and components capable of precisely measuring small voltage differences. The system further includes a processor 110 (e.g., computer processor) that runs a software application 114 designed to control the input variable, such as current amplitude and frequency. The software 114 can include preloaded test protocols, such as frequency sweep control, or may allow the operator to input custom test parameters. Further, the software 114 can acquire and process the voltage data in real time to compute the measured voltage phasor relative to the injected signal, across a frequency range of 1 kHz-1 GHZ.

The magnitude and phase angle data are visualized using graphical formats such as a Nyquist plot, Bode plot, or Cole-Cole plot, which enable the characterization of tissue types. The system is connected to a display unit 112 that provides real-time visual feedback on the tissue characteristics as the guidewires are positioned within the blood vessel.

### A. Guidewire Structure

Guidewires are thin, flexible wires that are used to navigate through blood vessels during endovascular procedures. Guidewires serve as a foundation for endovascular procedures, providing a pathway from outside the subject's body to the endovascular treatment location. Other devices such as a catheter, microcatheter, or stent can be advanced over the guidewire to access and treat the occluded blood vessels. Some guidewires may also have specialized functions depending on their intended use, for example, guidewires designed to cross chronic occlusions may have a much higher tip weight to allow passage through the calcified plaque. Some guidewires measure pressure most commonly to assess the hemodynamic significance of a coronary lesion/narrowing (fractional flow reserve pressure wires). Some of the commonly used guidewires for endovascular procedures are provided in Table 1.

TABLE 1
Types of guidewires used for endovascular procedures

| Type | Description |
|---|---|
| Standard & | Larger guidewire for passage through great vessels |
| Exchange | (0.035 inch) e.g., J-wire, Rosen, Bentson, Glidewire |
| Guidewires | |
| Stiff | Larger guidewire often for passage of structural |
| Guidewires | devices (0.035 inch) e.g., Amplatz Super Stiff, |
| | Lunderquist |
| Structural | Often with a preshaped curve e.g., Confida, Safari |
| Specific | |
| Guidewires | |
| Coronary | Usually 0.014 inch and includes: |
| Guidewires | Workhorse: Standard wire e.g., BMW, Sion Blue, |
| | Runthrough |
| | Hydrophilic: Pilot 150/200, Fielder XT |
| | Support: Grand Slam, Iron Man |
| | CTO (high tip load): Confianza Pro, Miracle Bros, |
| | Gaia |
| | Specialty: Rotawire (for rotablation), Pressure wire |
| | (FFR measurements) |
| Peripheral | Often 0.018 inch and includes: |
| Guidewires | Platform wires: V18, Glidewire, Spartacore |
| | Specialty: Supracore |
| Neurovascular | Often 0.014 inch, includes synchro, Trevo, Solitaire |
| Guidewires | |

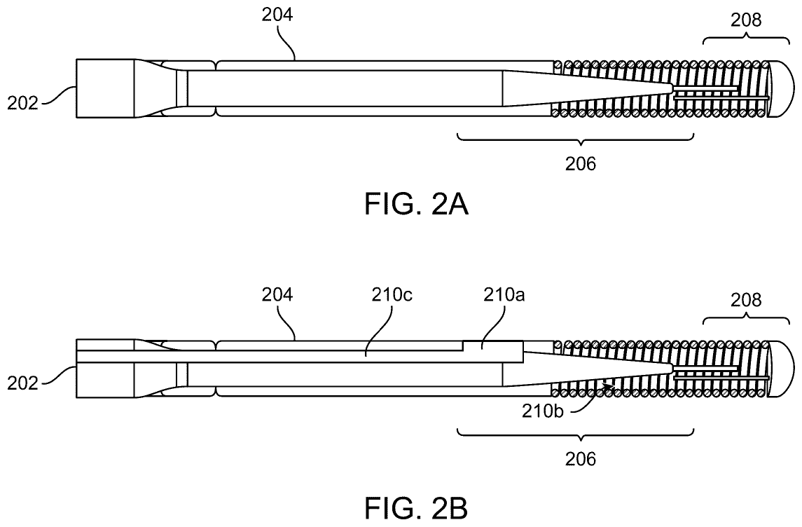

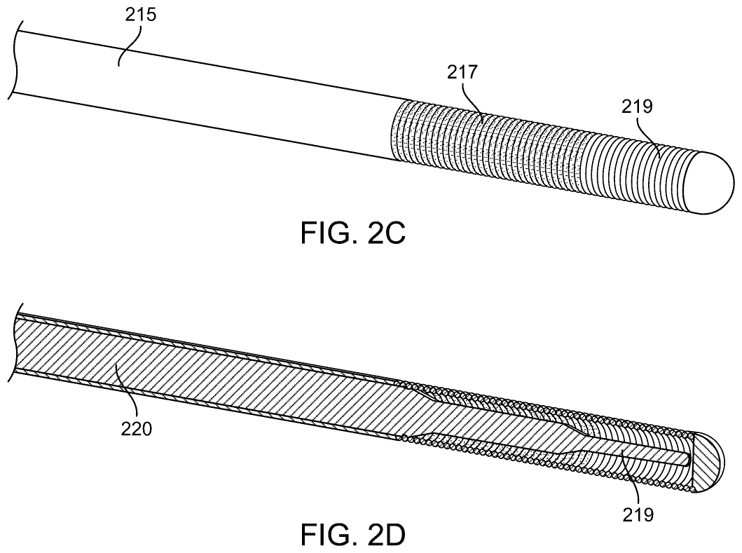

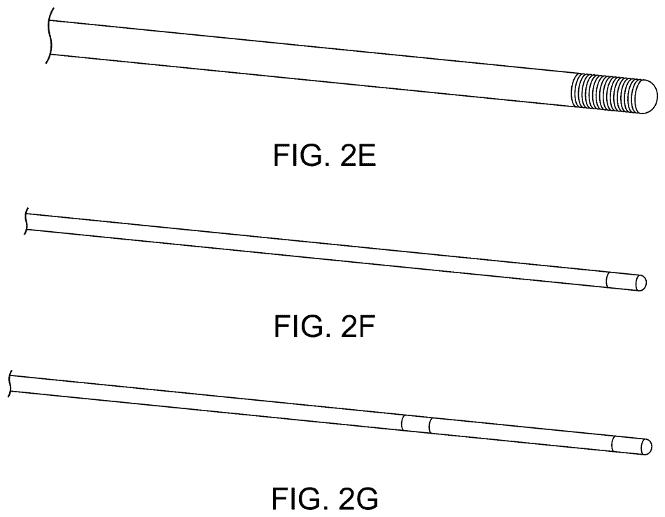

FIGS. 2A to 2G are schematic representations of example guidewire/elongated members without (2A) and with (2B-2E) modification. Guidewires possess mechanical properties that facilitate their movement through endoluminal pathways, with some wires having specific mechanical properties that aid in interventional procedural processes, for example crossing a CTO lesion in which case a wire with suitable stiffness and compressive resistance may be needed to cross hardened plaque tissue. The guidewire can be modified to include multiple tissue interfacing, conductive electrodes of various construction (i.e., coiled, conductive wire, ring electrodes), which connect via means of welding, soldering, thermal bonding, or adhesive bonding, to individual conductive members (e.g., wire, electrical tracks) which are electrically insulated from the surrounding environment through insulative layers or coatings (e.g., PTFE, PU, Silicone).

FIG. 2A represents a guidewire without modification. The guidewire includes a central shaft or core 202, which is made up of stainless steel or nitinol. Nitinol cores are more kink resistant but store torque, while stainless steel improves torque. Both these materials are conductive to electric current. The central shaft or core 202 is surrounded by core material 204, which is made up of coils or polymeric materials, such as polytetrafluoroethylene (PTFE). In some designs, the core material 204 is surrounded by coating material.

The core 202 includes a tapered region 206 which transitions into a distal flexible tip 208. The tip 208 includes a tight helix spring coil and is made from radio-opaque platinum or tungsten alloy, often coated with hydrophilic (e.g., polyethylene oxide or polyvinyl pyrrolidone) or hydrophobic (often PTFE or silicone) coating. The corrugated design of the tip 208 also provides tactile feedback to the user manipulating the wire. In some implementations, the tip 208 measures about 3-5 cm in length. The taper profile of the core 202 and the diameter of the tip 208 affect wire flexibility and support. For example, shorter core tapers may be prone to prolapse in tortuous blood vessels, while longer core tapers improve trackability.

FIG. 2C represents a guidewire with an elongated primary shaft 215 which includes an insulation region 217. To maintain acceptable mechanical performance, the distal tip from the insulation region 217 can extend further proximally from the distal electrode. This additional coil length may be conductive, carrying signal from the distal electrode, but electrically insulated from the body, or can be a separate component to the electrically conducting wire. The conductive electrode tip 219 may be exposed or can include a conductive wire or a single ring electrode component.

FIG. 2D represents a guidewire with a conductive inner member 220, which electrically couples the exposed distal tip 219 to the proximal end of the elongate primary shaft for direct connection to an external unit.

FIG. 2E represents an electrode which includes a single electrical coupling interface with an exposed conductive region on an elongated member. The electrode may exist at the distal tip or any position along the axial length of the member. The electrode can be constructed of exposed, coiled, conductive wire.

FIG. 2F represents an electrode which includes an elongated member made from individual ring electrode components. The ring electrode components are bonded to the elongated member via methods of swaging, welding, thermal bonding, or adhesive bonding.

FIG. 2G represents an electrode which includes an elongated member with multiple electrodes along the axial length. The electrode is connected to the proximal end of the elongated member via a conductive element.

In CTO procedures, the guidewire penetrates the tissue (plaque) of the complete occlusion and may involve higher penetrating power. This may be achieved by extending the core 202 to the tip 208 with variations in core profile and tip material, allowing variable tip pressure for any given tip stiffness (i.e., variable tip load). When combined with a reduced tip diameter, this provides high penetrating power against soft tissue.

Guidewires conduct electric current effectively due to the stainless steel or nitinol core. However, coatings such as PTFE, as well as surrounding devices like microcatheters or standard catheters, function as electrical insulators. In some designs, referring to FIG. 2B, the guidewire can have two conductive segments 210a and 210b with an insulation segment 210c in between them. One of the conductive segments can function as an active electrode and the other conductive segment can function as a sensing electrode. For example, one of the conductive segments can include a current injection electrode and the other a voltage sensing electrode. In another example, one of the conductive segments can include a voltage sensing electrode (WS) and the other segment can include the voltage reference electrode (R) separated by a fixed distance.

Most guidewires have exposed conductive segments of approximately 2-3 cm at both the proximal (external) and distal ends. In some designs, only the distal end (<1 mm) may be conductive, particularly if the distal coils are coated with hydrophobic PTFE. In contrast, if the coils are exposed or the coating is minimally insulative, electrical conductivity may extend proximally along the shaft. While the distal platinum coils may introduce minor inductive effects when current is applied, the guidewire behaves predominantly as a resistive conductor.

In some implementations, the system may include a specialized microcatheter configured to provide adjustable electrical insulation of the guidewire. Such a microcatheter may include, for example, a retractable insulating sheath or a series of individually addressable insulating segments. By actively controlling the insulation profile along the guidewire shaft, an operator can precisely define the length and location of the exposed electrode region during the procedure. This allows for fine-tuning of the measurement field and optimization of the sensing characteristics for different anatomical situations, providing an alternative means to control the electrical properties of the measurement system.

### B. Electrode Configurations

### 1. Bipolar Electrode Configurations

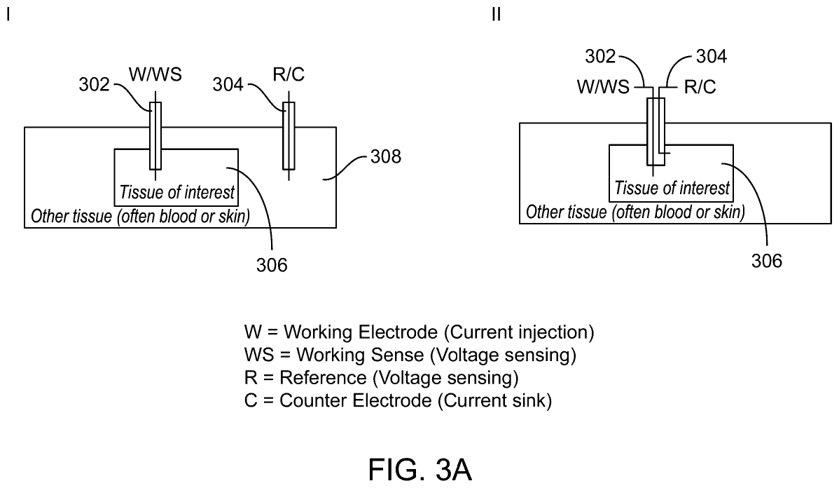

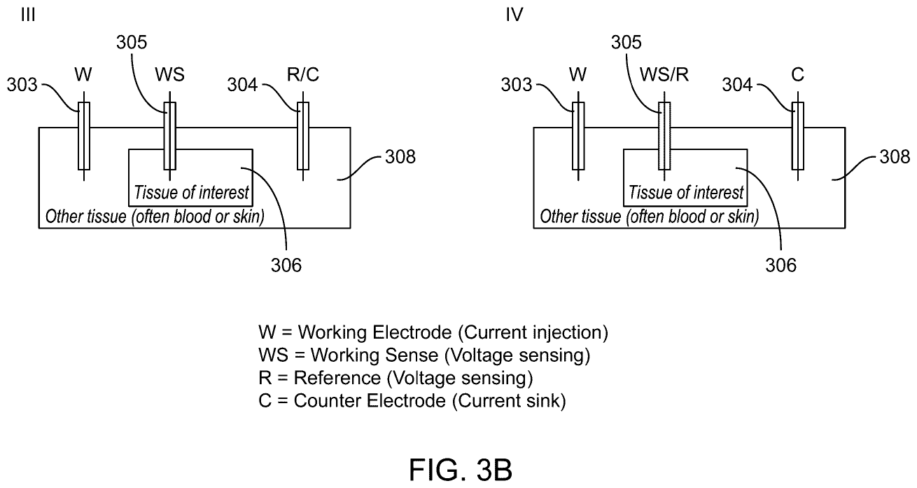

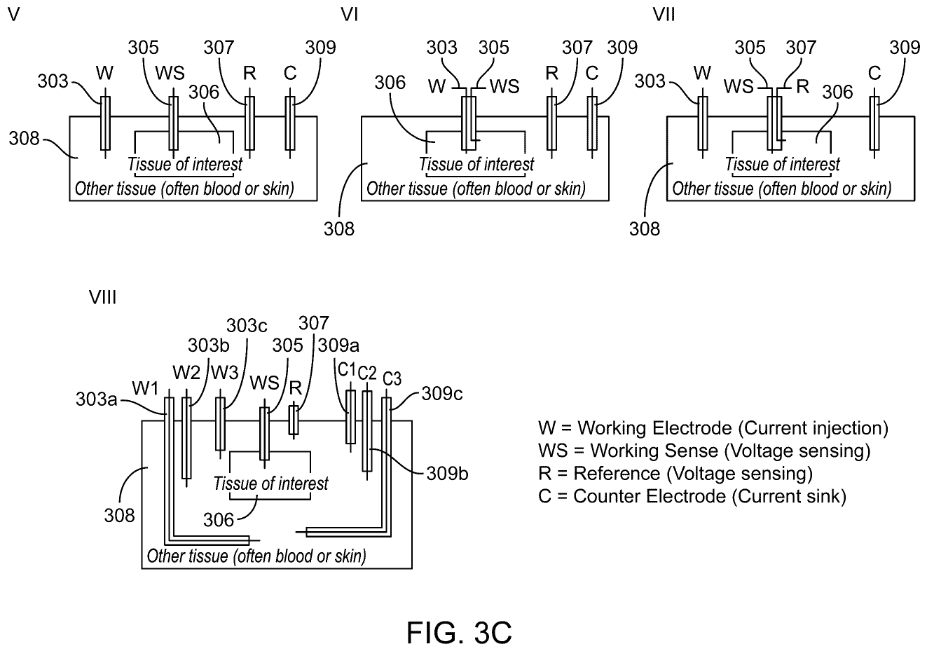

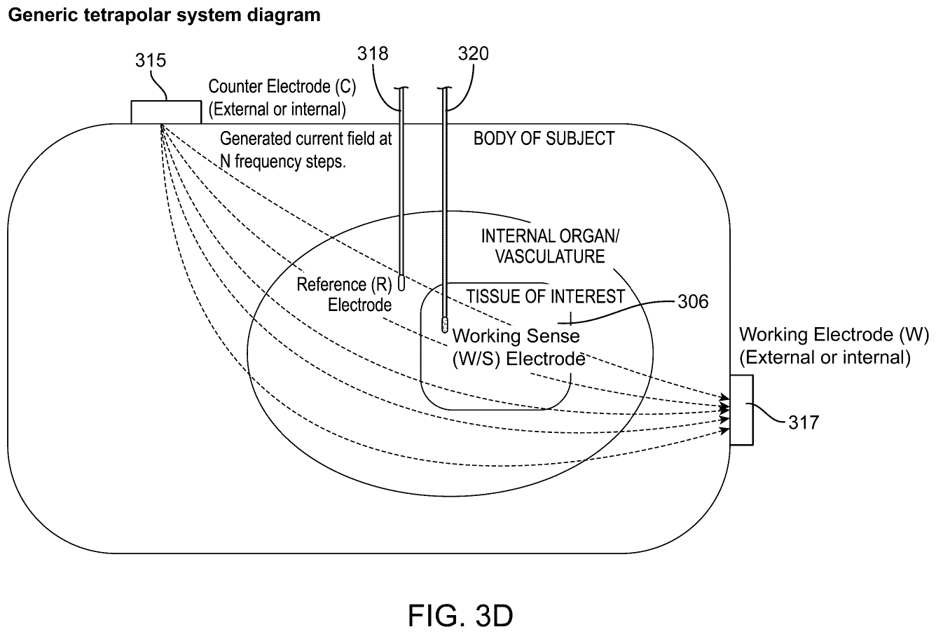

FIG. 3A is a schematic representation illustrating examples of bipolar electrode configurations. In an endovascular procedure (for example in a coronary CTO or peripheral CTO), guidewires are inserted into a blood vessel or the blood through small incisions or punctures. In case of coronary CTO procedures, the incisions are usually made at the wrist (radial artery) or groin region (right and left femoral artery or femoral vein). The radial or femoral arteries are preferred for accessing the coronary arteries because they are a large and easily accessible blood vessel that provides a direct route to the aorta, and these vessels are compressible against bony structures to prevent bleeding after removal of the catheters. From the aorta, the guidewire can be navigated by the operator into the desired coronary vessel. In case of peripheral CTO procedures, incisions are often made in the groin region (right and left femoral artery) to access the posterior tibial and peroneal arteries.

A bipolar configuration includes two electrodes to measure impedance. The electrodes can be positioned in different ways. Referring to FIG. 3A (configuration I), a first guidewire 302 is positioned within the tissue of interest 306. The first guidewire 302 includes both the current injection electrode (W) and the voltage sensing electrode (WS). The first guidewire 302 can be a modified guidewire with a dual conductive core. In addition to this, a reference electrode 304, which can include an external voltage sensing reference electrode (R) and/or a counter electrode (C), can be positioned in another tissue 308, such as within the blood or patched on the subject's skin. The working electrode within the tissue of interest 306 is a guidewire, while the electrode in the blood or on the skin can include a guidewire, a guidewire integrated into a catheter, or a skin patch electrode.

Referring to FIG. 3A (configuration II), the current injection electrode W and the voltage sensing electrode WS are included on the same guidewire 302, which is modified with dual insulated conductive cores. The guidewire 302 is positioned in the tissue of interest 306, such as a blood vessel, myocardial space, subintima, or fat. In addition, the counter and/or reference electrodes R and C are included on a second modified guidewire 304 and can be positioned in the same tissue of interest 306. Both the guidewires measure voltage magnitude and phase angle. As described earlier, in some implementations, an external electrode is placed on the subject's skin surface.

### 2. Tripolar Electrode Configurations

FIG. 3B is a schematic representation illustrating examples of tripolar electrode configurations. In a tripolar configuration, three electrodes are used to collect impedance-based measurements in a tissue. In configuration III, a first electrode 303 includes the current injector electrode W and is positioned in a tissue 308, such as within the blood or patched on the skin. The first electrode 303 can include an external electrode, a guidewire, or a guidewire integrated on a catheter. A guidewire 305 functions as the voltage sensing electrode WS and is placed in the tissue of interest 306. A third electrode 304 functions as both the reference and counter electrodes R and C. The third electrode 304 can be an external skin patch electrode or a guidewire placed in the tissue 308, such as within the blood or patched on a skin. In some implementations, the third electrode 304 can be placed in another tissue (e.g., a blood vessel) adjacent to WS.

In configuration IV, a first electrode 303 includes the current injector electrode W and is positioned in a tissue 308, such as within the blood or patched on the skin. A guidewire 305 functions as both the voltage sensing electrode WS and the reference electrode R and is placed in the tissue of interest 306. The guidewire 305 in initially placed in blood during a single sweep measurement and the baseline value obtained is used as the reference value R. In some implementations, the guidewire 305 can be a modified guidewire with a dual conductive core, where the conductive cores are separated by an insulation. In this case, the guidewire 305 with the modification can function as both WS and R. A third electrode 304 functions as the counter electrode C, which can be placed in the tissue 308, such as blood or on the skin.

In some implementations, a first electrode 303 includes the current injector electrode W and is positioned in a tissue 308, such as within the blood or patched on the skin. A guidewire 305 functions as the voltage sensing electrode WS, placed in the tissue of interest 306. During each measurement sweep, the input current alternates between a high and low value via a parallel resistor. The measured voltage difference between the alternating cycles is used to calculate the potential difference between WS and R. A third electrode 304 functions as the counter electrode C, which can be placed in the tissue 308, such as blood or on the skin.

### 3. Tetrapolar Electrode Configurations

FIG. 3C is a schematic representation illustrating examples of a tetrapolar electrode configuration. A tetrapolar EIS system includes four electrodes to characterize tissue with impedance-based measurement. In configuration V, a first electrode 303 functions as the current injector electrode (W). W can include an external electrode placed in a tissue 308, such as in blood or on the skin. A guidewire 305, placed in the tissue of interest 306, functions as the voltage sensing electrode (WS). A reference electrode (R) 307 and a counter electrode (C) 309 are placed in the tissue 308.

In configuration VI, the first electrode 303 (W) and the guidewire 305 (WS) can be integrated on a modified/custom guidewire with two insulated conductive cores. In some implementations, a microcatheter that includes two guidewires W and WS can be used. Both W and WS are placed in the tissue of interest 306. The reference electrode (R) 307 and a counter electrode (C) 309 are placed in the tissue 308, such as in blood or on the skin.

In configuration VII, the first electrode 303 functions as the current injector electrode (W) and counter electrode 309 functions as the current sink C. W and C are placed in the tissue 308, such as in blood or on the skin. W and C generate an electric field that transmits through blood mostly and passes into the tissue of interest 306. The guidewire 305, which functions as the voltage sensing electrode WS, and the reference electrode 307 (R) are placed in the tissue of interest 306. The guidewire 305 and the reference electrode 307 can be two separate guidewires or can be integrated on a modified guidewire with a dual conductive core. In some cases, WS and R are placed in a microcatheter.

In configuration VIII, a multiport 4-electrode system is used to generate a 3D positional information with two or more C/W pairs. For example, multiple current injector electrodes W 303a, 303b, 303c are placed in the tissue 308, such as in the blood or on the skin. Multiple counter electrodes C, such as 309a, 309b, 309c are placed in the tissue 308. The W/C pairs generate an electric field across the region of interest, mostly in the blood stream. The guidewire 305 (WS) is placed in the tissue of interest 306, such as a coronary vessel or a peripheral vessel, or any endovascular tissue. The reference electrode 307 (R) is placed in the tissue 308. In some implementations, the reference electrode R 307 is placed in the tissue of interest 306. A voltage difference across WS and R is measured for calculating the apparent impedance or the dielectric response characteristics of the tissue of interest 306.

In case of a coronary CTO, W and C can include skin patches (e.g. Ag/AgCl) positioned on the chest (e.g., right side of the neck and left mid-axillary line), while WS and R are intravascular guidewires with exposed tips inserted into the vasculature. In some implementations, R can be a skin electrode. In other implementations, R can be a second electrode integrated into a modified/custom guidewire with two insulated conductive cores, such that WS and R are on the guidewire tip. In other implementations, a modified/custom guidewire with two insulated conductive cores can be configured with W and WS at the wire tip and with C and R as separate guidewires, a catheter tip, or skin electrodes.

When a low AC current (e.g., 10 μA-10 mA, 1 kHz-10 MHZ) is applied through W and C, the voltage difference between WS and R is measured to calculate apparent imaginary and real impedance. When current is not being directly injected or sunk through the tissue of interest, the system measures the voltage phasor relative to the input signal in different tissues, as a surrogate, rather than true complex impedance. In some implementations of a 4-electrode configuration, the current carrying electrodes W and C create an electric field within the body, with most current passing through the blood stream and lesser through the subintima, myocardium, pericardial space, or plaque. This electric field represents the region where the current flows, influenced by the conductivity and geometry of the tissues. For example, geometric factors, such as the distance between W and C, the axial and lateral positions of WS and R, their relative angles, and their tissue locations can significantly influence the electrical impedance measurements. In implementations for endovascular CTO interventions, the electrical impedance measurements are used to distinguish between blood, plaque, vessel wall, myocardium, and pericardial space, without altering standard equipment or clinical workflows.

FIG. 3D is a schematic representation of an exemplary tetrapolar system. A low amplitude alternating current field is generated between the counter electrode C 315 and working electrode W 317 across the body of the subject, internal organs and vasculature and the tissue of interest 306. The reference electrode (R) 318 is placed at a location within the internal organs or vasculature or on the skin. A working sense (WS) electrode 320 is placed in the tissue of interest 306. Voltage magnitude and phase are measured between WS and R and apparent impedance calculated to infer the tissue type surrounding the WS electrode.

### 4. Exemplary Reference and Working Sense Electrode Locations in a Heart Muscle

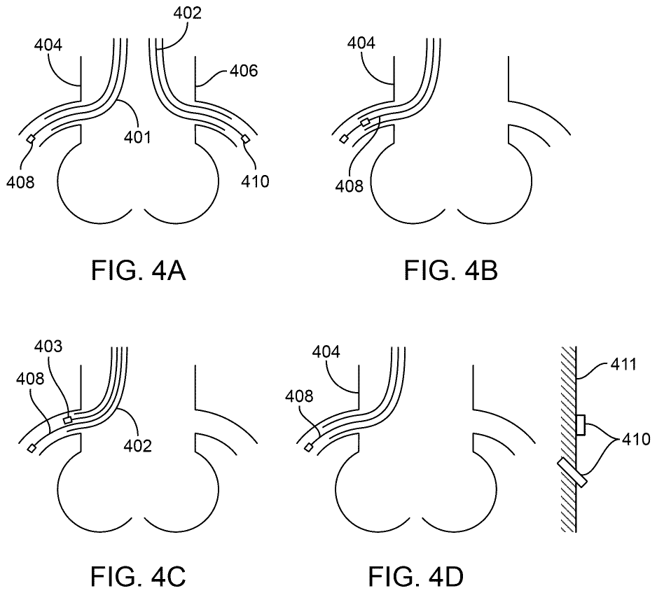

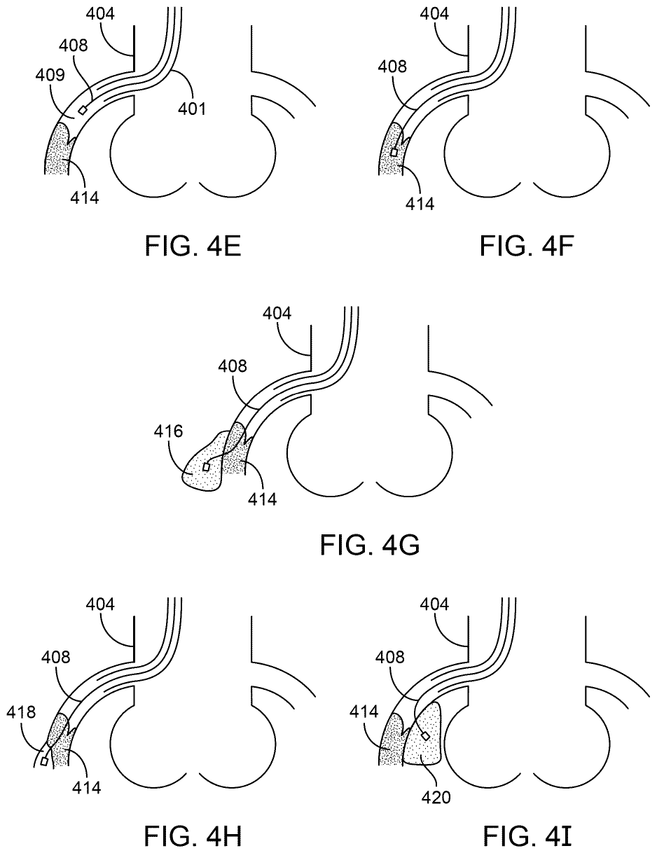

FIGS. 4A, 4B, 4C, 4D, 4E, 4F, 4G, 4H, and 4I are schematic representations illustrating examples of common positions for reference (R) electrodes and working sense (WS) electrodes within or nearby a coronary vessel.

In FIG. 4A, a catheter includes a guidewire positioned within one of the coronary vessels (e.g., right coronary artery 404 or left coronary artery 406). In the configuration illustrated in FIG. 4A, a first catheter 401 includes the first guidewire 408 and is positioned in the right coronary vessel 404 (the tissue of interest) and functions as WS. A second catheter 402 includes a second guidewire 410, which is positioned in the left coronary vessel 406. The second guidewire 410 functions as R. The first guidewire 408 and second guidewire 410 can also function as W and C, for example in a two electrode configuration, or may only act as WS and R, for example in a four electrode configuration.

In FIG. 4B, a single guidewire 408, which includes both the voltage sensing (WS and R) electrodes, is placed in a single coronary vessel, for example, in the right coronary vessel 404. The guidewire 408 can also be placed in the left coronary vessel or any other blood vessel or lumen. In some implementations, a catheter includes a shaft through which the guidewire 408 is inserted. In this configuration, the guidewire 408 is a modified guidewire with two insulated conductive cores with the two electrodes on the guidewire tip acting as WS and R. In some cases (e.g., two-electrode configuration), these electrodes also may act as W and C. The insulation may be provided by a PTFE coating.

In FIG. 4C, a catheter 402 includes an electrode which is integrated into the catheter tip 403. The catheter 402 along with an integrated electrode is placed in the right coronary vessel 404. A guidewire 408 functions as WS and the catheter tip 403 electrode functions as R. These electrodes may also act as W and C, for example in a two-electrode configuration. In this configuration, the length of the guidewire 408 can be varied to create a specific linear distance between the current injector W and sensing electrode WS. This allows for effective current field transmission through the blood stream, facilitating accurate impedance-based measurement. This configuration is useful when a custom catheter is used.

In FIG. 4D, a first guidewire 408 functions as the sensing electrode WS and is placed in a single coronary vessel, such as the right coronary vessel 404. The reference electrode 410 (R) is positioned on the skin 411. The two electrodes may act as W and C, for example in a two-electrode configuration. The skin reference electrode 410 can include an Ag/AgCl skin patch, which has low impedance and a stable half-cell potential. The skin patch electrode includes a conductive gel, which provides a reliable connection to the skin. This minimizes motion artifacts and ensures high quality signal transmission. This configuration is used when a second guidewire is not required.

In FIG. 4E, a first guidewire 408 is positioned in the right coronary vessel 404 and functions as WS electrode (and sometimes the W electrode, e.g., in a two-electrode configuration). The tip of the guidewire 408 (e.g., 1-3 cm) is exposed and is in contact with the tissue in which it is placed. The right coronary vessel 404 is at least partially occluded by plaque 414. The first guidewire 408 is placed in the blood stream 409 (tissue in contact) of the right coronary vessel 404, just above the occluded region and impedance-based measurements are obtained (e.g., voltage potential magnitude and phase). The reference electrode (R) can be placed at a location shown in 4A-D.

When a low amplitude AC current (1 μA-1 mA, 1 kHz-1 MHZ) is applied via W and C (positions not shown), a current field is generated and it is transmitted across the region of interest, mostly via the blood stream (acts as the electrolyte) 409. A voltage difference is measured across two electrodes (WS-shown, and R-location not shown), which is used to determine the real and imaginary components of apparent impedance. Additionally, the phase shift between the input electrical signal and the output signal is measured, providing insight into the impedance characteristics of the tissue in contact with the first guidewire 408, i.e., blood stream 409 in this case. In a 2-electrode configuration, the measured impedance value for a blood stream is about 150-500 ohms (Ω)

In FIG. 4F, the first guidewire 408 is positioned in the plaque 414 of the right coronary vessel 404 and functions as WS. The tip of the first guidewire 408 is exposed and is in contact with the plaque 414. A second electrode (not shown) functions as R.

When a low amplitude AC current is applied via W and C (positions not shown), a current field is generated and it is transmitted across the region of interest, mostly via the blood stream (acts as the electrolyte). The voltage difference across the sensing electrode WS and reference electrode R is measured to determine the apparent impedance value. A Nyquist plot, Bode plot, or Cole-Cole plot is used to visualize the impedance data and characterize the tissue type in contact with the first guidewire 408. In a 2-electrode configuration, the plaque 414 exhibits an impedance value in the range of about 1000-3000 Ω.

In FIG. 4G, the first guidewire 408 is positioned outside the right coronary vessel 404, for example, in the pericardial space 416 (which can include a perforation outside the vessel). It functions as WS electrode. The right coronary vessel 404 is at least partially occluded by plaque 414. The tip of the guidewire 408 is exposed and is in contact with the pericardial space 416. A second electrode (not shown) functions as the R electrode.

When a low amplitude AC current is applied via the W and C electrodes (positions not shown), a current field is generated and it is transmitted across the region of interest, mostly via the blood stream (acts as the electrolyte). The voltage difference across the sensing (WS) and reference (R) electrodes is measured to determine the apparent impedance value. A Nyquist plot, Bode plot, or Cole-Cole plot is used to visualize the impedance data and characterize the tissue type in contact with the first guidewire 408. In a 2-electrode configuration, the blood vessel wall (pericardial space) exhibits an impedance value in the range of about 500-1000 Ω.

In FIG. 4H, a first guidewire 408 embedded in the wall of the right coronary vessel 404, for example, in the subintimal space 418 (which can include a perforation in the vessel wall). It functions as WS. The right coronary vessel 404 is at least partially occluded by plaque 414. The tip of the guidewire 408 is exposed and is in contact with the subintimal space 418. A second electrode (not shown) serves as the reference electrode (R). On applying a low amplitude AC current via the W and C electrodes (positions not shown), a current field is generated and it is transmitted across the region of interest, mostly via the blood stream (acts as the electrolyte). The voltage difference across the two electrodes (WS and R) is measured to determine the apparent impedance value. In a 2-electrode configuration, the coronary subintimal space exhibits an impedance value in the range of about 300-800 Ω.

In FIG. 4I, a first guidewire 408 is embedded in the myocardium 420, after perforating through the right coronary vessel 404. It functions as WS. The right coronary vessel 404 is at least partially occluded by plaque 414. The tip of the guidewire 408 is exposed and is in contact with the myocardium 420. A second electrode (not shown) serves as the reference electrode (R). On applying a low amplitude AC current via the W and C electrodes (positions not shown), a current field is generated and it is transmitted across the region of interest, mostly via the blood stream (acts as the electrolyte). The voltage difference across the two electrodes (WS and R) is measured to determine the apparent impedance value.

In these examples, common positions for WS and R electrodes are shown. These positions may be implemented with two (bipolar), three (tripolar), or four electrode (tetrapolar) configurations, where the W/C electrodes may be different electrodes to WS/R, or where WS/R electrodes may serve as both WS/R and W/C electrodes. The tetrapolar approach is well-suited for applications where mitigating the effects of electrode-tissue interface impedance is desired.

Exemplary coronary CTO and peripheral CTO tetrapolar configurations

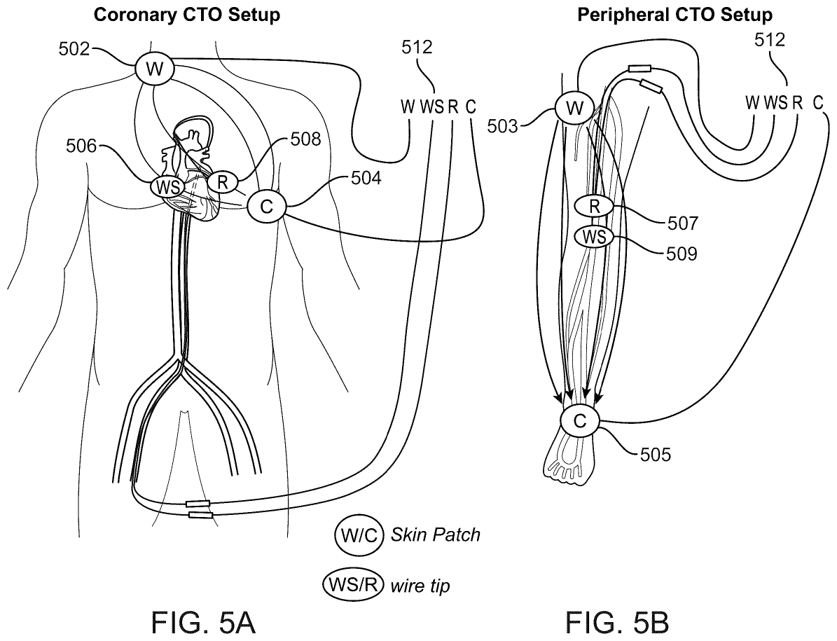

FIGS. 5A and 5B are schematic representations of exemplary tetrapolar configurations in a coronary CTO intervention and a peripheral CTO intervention, respectively. In FIG. 5A, the current carrying electrodes 502 (W) and 504 (C) are positioned externally on the subject's skin surface. The current carrying electrodes can include skin patch electrodes, such as Ag/AgCl. A conductive gel is used to patch the electrodes to the skin. The current carrying electrode 502 (W current injector) can be positioned on the right or left side of the neck (anterior position) or on the surface of the chest. Electrode 504 is placed at a sufficient axial distance from W to generate an electric field that spans the chest volume. A low AC current is supplied from a signal generator device 512 to the current carrying electrodes 502 and 504. The blood stream acts as the electrolyte and transmits the generated current field in the chest region.

Two intravascular guidewires 506 (WS) and 508 (R) are placed in the path of the generated current field. Each guidewire has an exposed tip and has a long flexible arm with a spindle attached for easy rotation during the intravascular intervention. For an endovascular procedure, such as a coronary CTO procedure, guidewires are positioned in the blood vessel occluded by plaque by making an incision at the wrist (radial artery) or groin region (right and left femoral artery). The radial or femoral arteries are preferred for accessing the coronary arteries, because they are large and easily accessible blood vessels that provides a direct route to the aorta and subsequently to the coronary blood vessel of interest (e.g., occluded by plaque).

The guidewire 506 (WS) is positioned, for example in the left descending artery, right coronary artery, left circumflex, or in any other coronary vessel, which may be occluded with plaque or fat. The guidewire 508 (R) is placed either in the same coronary vessel as WS or can be placed in another coronary vessel or could be a separate electrode, for example on the skin. The positioning of the guidewire 508 is determined by the operator based on a combination of factors, such as workflows and geometric factors that affect impedance measurements. As the guidewires are being positioned in the coronary vessels, an imaging technique, such as a fluoroscopy (x-ray) is used to navigate and confirm the placement of the guidewires in the coronary vessel. The imaging techniques provide a projection of the guidewire (and catheter). In some implementations, radio-opaque iodine contrast can be injected down the coronary vessel (or any vessel of interest) to show a 2D projection of the lumen of the vessel and the X-ray unit is used to take images from multiple angles. The spatial resolution of fluoroscopy is <0.2 mm and images are commonly taken at 15 frames per second.

Once the guidewires 506 and 508 are positioned in the coronary vessels, a low amplitude AC current is applied to the current carrying electrodes. A current field is transmitted across the chest spanning from C to R. The guidewires 506 and 508 are conductive due to their core material. A voltage difference between the guidewires WS and R is measured by the measurement unit (e.g., a potentiostat) device 512. A processor (e.g., computer system) that includes a software application, processes the voltage output data to calculate apparent impedance based on the following equations:

$$Z = \frac{{V}_{diff}}{I} \qquad ( Equation 1 )$$

$${Z}_{real} = \frac{\vert {V}_{1} \vert cos ( {ϑ}_{V 1} ) - \vert {V}_{2} \vert cos ( {ϑ}_{V 2} )}{\vert I \vert} \qquad ( Equation 2 )$$

$${Z}_{imag} = \frac{\vert {V}_{1} \vert sin ( {ϑ}_{V 1} ) - \vert {V}_{2} \vert sin ( {ϑ}_{V 2} )}{\vert I \vert} \qquad ( Equation 3 )$$

$$\vert Z \vert = \sqrt{{Z}_{real}^{2} + {Z}_{imag}^{2}} \qquad ( Equation 4 )$$

$$Θ = arctan ( \frac{{Z}_{real}}{{Z}_{imag}} ) \qquad ( Equation 5 )$$

$${V}_{diff} = {V}_{1} - {V}_{2} \qquad ( Equation 6 )$$

where Z is the impedance, Zᵣₑₐₗ is the real component of impedance (resistance), Z_{imag} is the imaginary component of impedance (reactance), θ is the phase angle or the phase difference between the output voltage and the input current, q is phase angle of the recorded signals relative to the input I, I is the current amplitude in amperes, Vdiff is the voltage difference, V₁, the voltage on WS, and V₂, the voltage on R, are the voltage of the guidewires.

In some cases, the frequencies representing tissue properties may be plotted and calculated, for example, when Zᵣₑₐₗ and Z_{imag} are equal, representing the frequency when conduction through the tissue transitions from ionic to dielectric (i.e., when the phase angle at WS is negative 45 degrees).

A Nyquist plot, a Cole-Cole plot, or a Bode plot are used to visualize the impedance measurements for each tissue type. The unique trace of these plots is used to distinguish between the different tissue types, such as blood, plaque, vessel wall, myocardium, and pericardial space during the CTO procedure, without modifying any workflows.

FIG. 5B relates to a peripheral CTO intervention. Similar to the procedure described in FIG. 5A, the current carrying electrodes 503 (W) and 505 (C) are positioned externally on the subject's skin surface, for example on the surface of the leg in this configuration. The current carrying electrodes 503 and 505 can include skin patch (e.g., Ag/AgCl) electrodes. In some implementations, the current carrying electrodes can include guidewires with an exposed tip and placed intravascularly. For an endovascular procedure, such as a peripheral CTO procedure, the intravascular guidewires 507 (WS) and 509 (R) with an exposed tip are placed in the blood vessel of the leg, for example the femoral artery, which can be occluded with plaque. In some implementations, WS and R are placed in the blood, vessel wall, or muscle. The electrodes WS and R are positioned in the path of the current flow generated by W and C. As the intravascular guidewires 507 and 509 are being positioned in the blood vessel of the leg, fluoroscopy imaging techniques, as described previously, are used to navigate, and confirm the placement of the guidewires.

After positioning the guidewires, a low amplitude AC current is applied to the current carrying electrodes 503 and 505 by the signal generator device 512. The current field is transmitted through the blood stream. A voltage difference across the sensing electrodes WS and R is measured by the measurement unit 512. The voltage output data is used to calculate apparent impedance and the phase angle based on Equations 1-6. A Nyquist plot, a Cole-Cole plot, or a Bode plot are used to visualize the impedance measurements for each tissue type and characterize the tissue type during the CTO procedure.

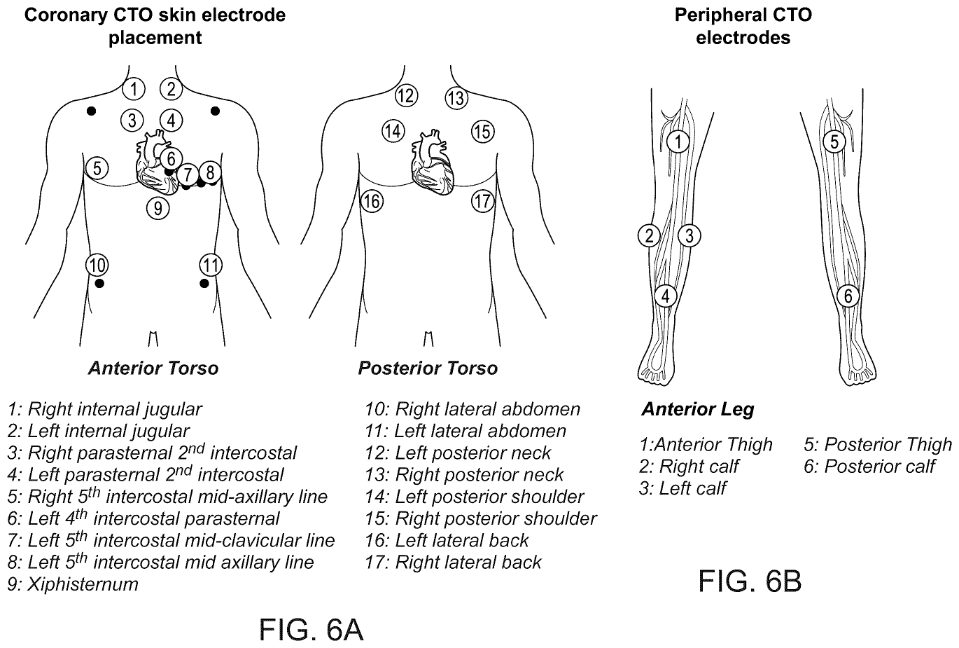

FIGS. 6A and 6B are schematic representations of example skin electrode placements for a coronary CTO intervention and a peripheral CTO intervention, respectively. For a coronary CTO (FIG. 6A), the skin electrodes W/C can be positioned at various locations, such as near the right or left internal jugular, right or left parasternal 2nd intercoastal, right 5th intercoastal mid-axillary line, left 4th intercostal parasternal, left 5th intercostal mid-clavicular line, left 5th intercostal mid axillary line, xiphisternum, or right or left lateral abdomen. For a peripheral CTO (FIG. 6B), the skin electrodes W/C can be positioned at anterior thigh, right or left calf, posterior thigh, or posterior calf.

These electrodes may be configured as a single set of W/C electrodes or as multiple electrodes sets. The multiple W/C sets generate multiple current axes, enabling precise localization of each tissue and characterization of each tissue using real voltage magnitude (where a real voltage gradient is generated between each C1/C2 electrode pair).

### C. Effect of Electrode Geometries on Impedance-Based Measures

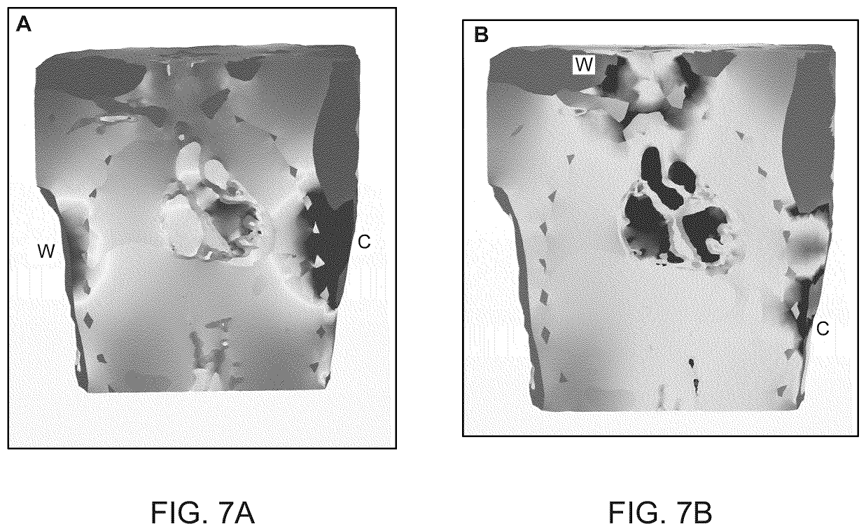

FIGS. 7A and 7B are schematic representations illustrating current density based on the placement of W and C in a torso region. Current density (current per unit area) varies depending on the electrodes W and C placement and the applied frequency. FIG. 7A shows placement on the lateral chest wall, while FIG. 7B depicts placement via the neck and lower left abdomen. These examples demonstrate that current density is influenced by electrode placement, tissue conductivity, and permittivity. Most of the current conducts via the blood stream, with lesser portions passing through the subintima, myocardium, pericardial space, or plaque.

In some cases, increasing the distance between W and C, the current carrying electrodes, affects the current field and impedance measurements in several ways. A larger W to C distance increases the total impedance of the current path by incorporating more tissue. For example, if the distance between W and C doubles (e.g., from 20 cm to 40 cm), the impedance of a homogeneous medium doubles. This increases the voltage drop across the path. A larger distance spreads the current over a wider volume, reducing current density along the axial path. This can decrease measurement sensitivity to local tissue changes between WS and R, as the current field becomes more diffuse.

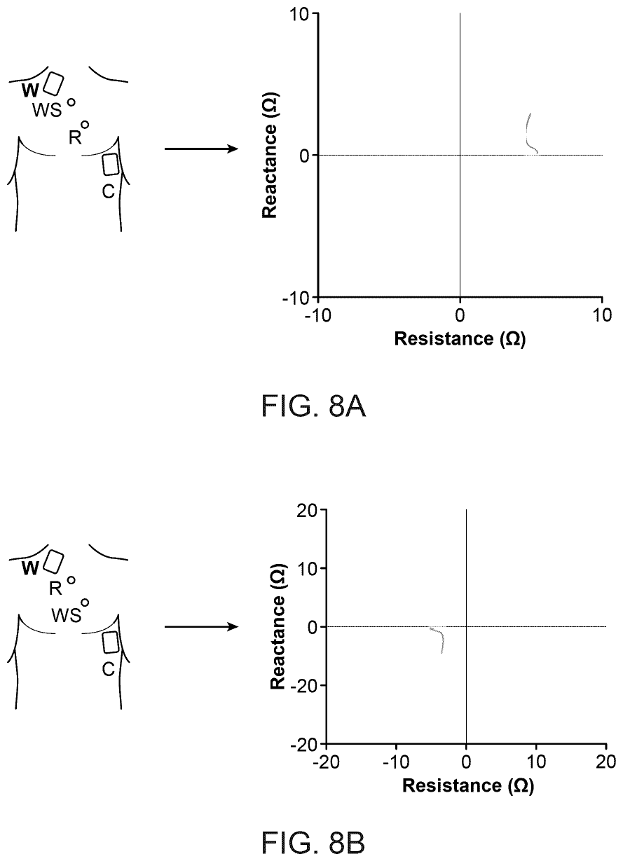

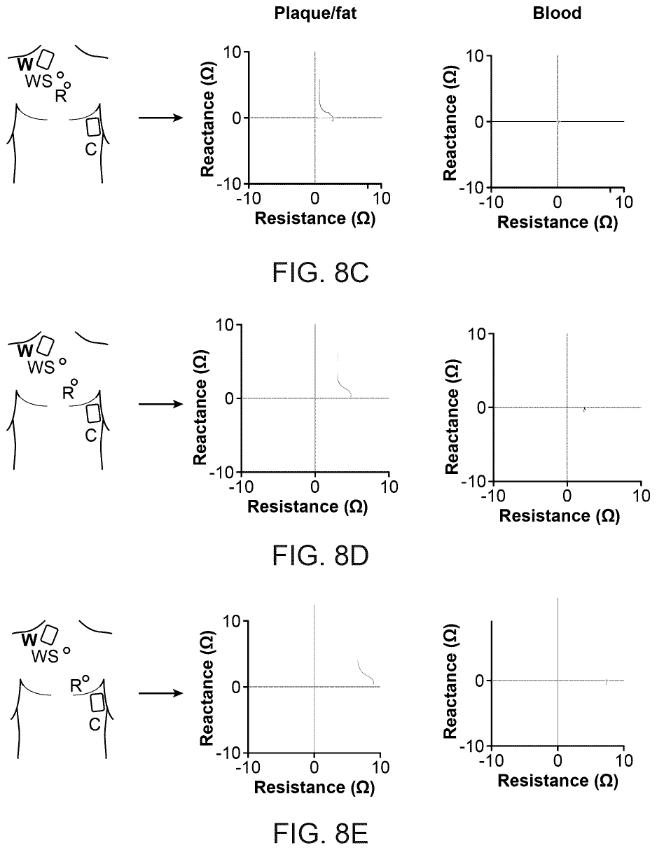

FIGS. 8A, 8B, 8C, 8D, and 8E are schematic representations illustrating the effect of electrode placement on impedance measurements. In a 4-electrode setup, WS and/or R may be mobile in the electric field if configured as a guidewire, as the guidewire navigates through a tissue during a procedure. During this process, if WS is closer to W, the real impedance and imaginary impedance (in the presence of a capacitive tissue) will be positive as shown in FIG. 8A. Whereas if WS is closer to C (i.e., if it moves past R during the procedure), then the real and imaginary impedance will be negative, as shown in FIG. 8B. If negative impedance is obtained, it can be adjusted during signal processing.

FIGS. 8C-8E illustrate exemplary electrode configurations, where W and C inject current via skin electrodes, and R moves through the tissue of interest, while WS functions as a reference in blood. In FIG. 8C, R is close to WS. In FIG. 8D, R is mid distance from WS, and in FIG. 8E, R is far away from WS (these movements may occur in a procedure as the guidewire tip moves through a vessel). The distance between WS and R alters the apparent impedance (Z) real measurement due to the geometry of the electric field, rather than tissue properties. However, the Z imaginary measurement can consistently characterize the difference in tissue types, such as blood, plaque, vessel wall, subintima, or muscle (i.e., the trace of the tissue is consistent for each tissue type). The apparent Z real value could be normalized to center the measurements on the plot (i.e., place at an average value of 0 on the x-axis). In some cases, the apparent Z real value could be used for positional information in 3D plotting, if multiple axes are used.

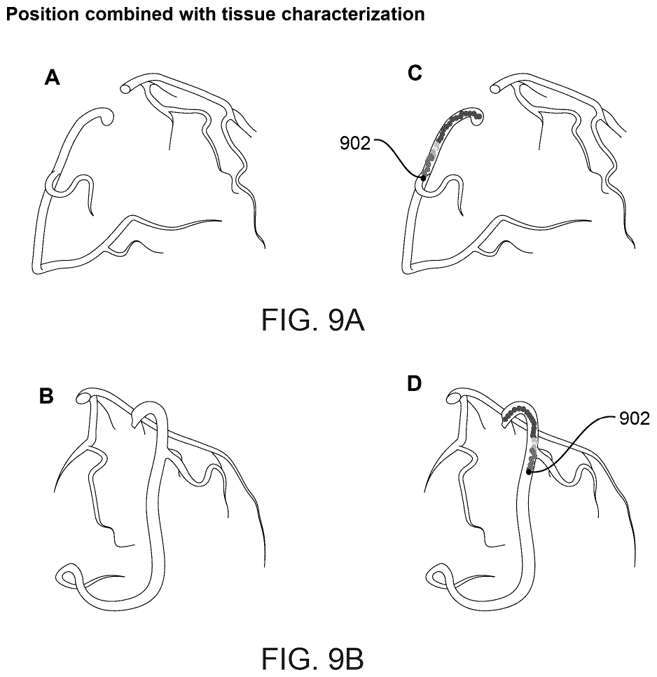

FIGS. 9A-9B illustrate 3D representations of the coronaries, based on a segmentation from a computed tomography data, for a right anterior oblique and left anterior oblique view, respectively, to show an example of a guidewire position in a vessel. In some implementations, multiple ports allow the system to create overlapping current fields along different axes (e.g., transverse, vertical, diagonal) by alternating current injection between different W-C pairs. In some implementations, the system performs a multi-frequency sweep (e.g., 1 kHz-1 MHZ) for each current field, calculating impedance for each current axis. A multi-current field can reduce any voltage measurement ambiguities arising due to the geometric placement of the sensing electrodes.

The system can track the dielectric properties of the tissue in contact with the guidewire tip by plotting the apparent Z imaginary values or phase angles (averages or specific frequencies), according to the voltage magnitude. With a single axis (one port with one pair of W/C) a linear plot could be generated, with two axes (two ports with two pairs of W/C) a 2D representation could be generated, and with three axes (three ports with three pairs of W/C) a 3D representation could be generated. The shaded portion in FIGS. 9A and 9B represent the average apparent Z imaginary magnitude, while the black point 902 indicates the current position of the guidewire. 3D representations could be fused with angiography/fluoroscopy imaging, intravascular imaging, or pre-procedural imaging such as computed tomography.

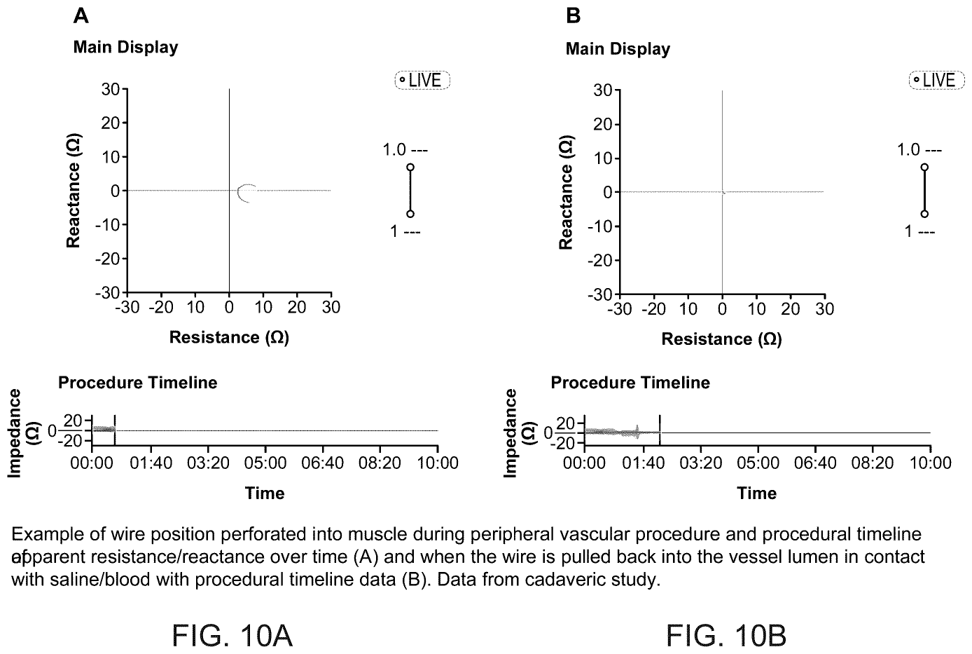

FIGS. 10A and 10B illustrate an example of a guidewire position during a peripheral vascular procedure, during a cadaver study. In FIG. 10A, a guidewire is initially placed in a peripheral artery, and perforated through the vessel wall into the muscle during the procedure. The position and the procedural timeline of the apparent resistance/reactance can be observed. FIG. 10B represents the resistance/reactance over time when the guidewire is pulled back into the vessel lumen and is in contact with saline/blood.

### D. Preclinical Studies-Coronary CTO Interventions

In some implementations, the disclosed systems and methods may be applied to support the performance of CTO interventions in coronary arteries. These procedures involve navigating guidewires through fully occluded segments of coronary vessels, where conventional visualization techniques may offer limited utility. To evaluate the feasibility and performance of impedance-based tissue characterization in this context, preclinical studies were conducted using an animal model to simulate human coronary CTO interventions and complications of these procedures. The following paragraphs describe the experimental setup and electrode configurations used in those studies.

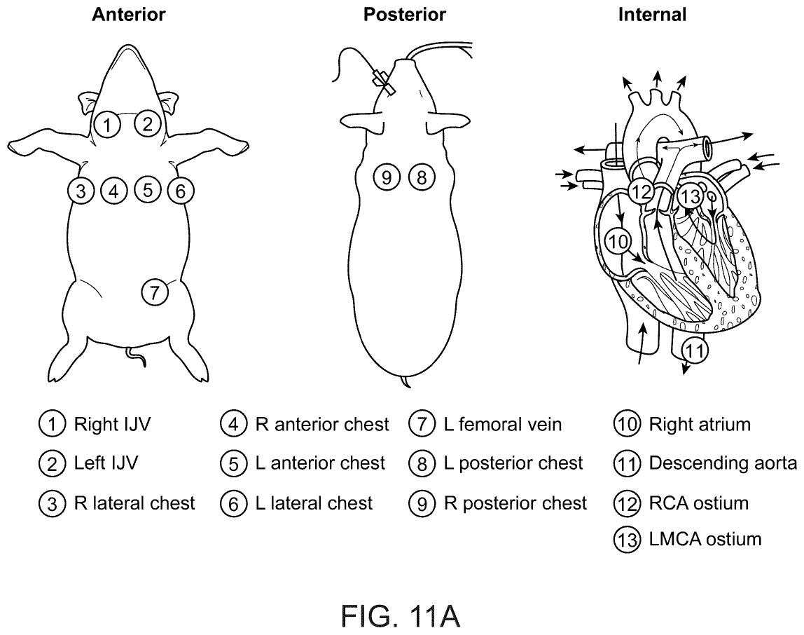

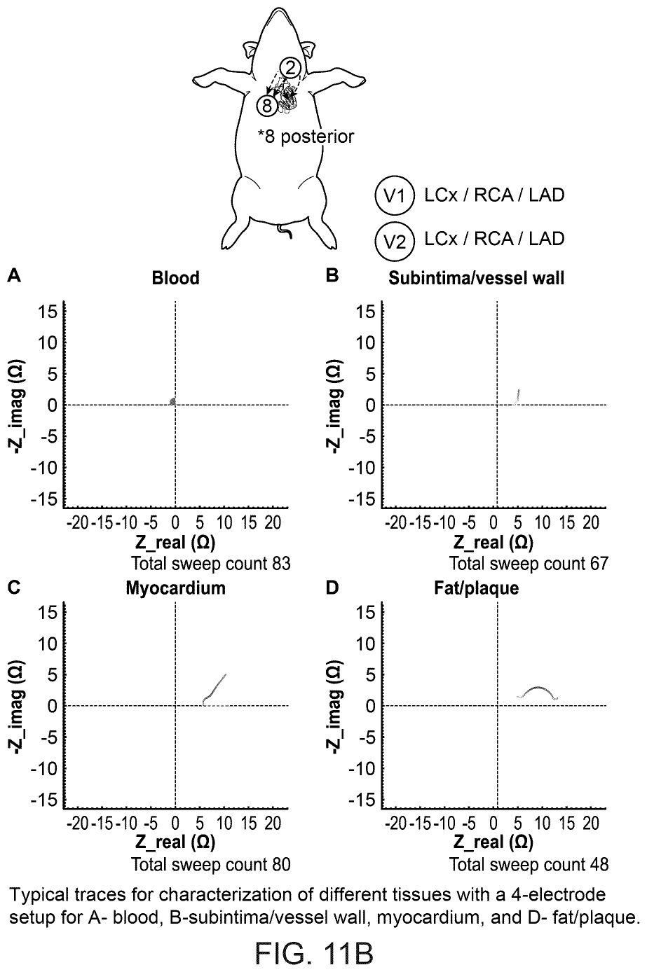

FIG. 11A is a schematic representation illustrating the anatomical positions of the subject's skin and cardiac system for a 4-electrode placement for EIS measurements. For performing a coronary CTO intervention, a porcine of about 40-50 kg was selected for the preclinical studies. The porcine was intubated and placed under general anesthesia prior to the CTO intervention. A tetrapolar electrode configuration was chosen for the procedure. For the coronary CTO intervention, Ag/AgCl skin electrodes were positioned externally on the skin's surface as the current carrying electrodes W and C. Intravascular coronary guidewires were used as the sensing electrodes WS and R. The coronary guidewires were selected from, for example, Astato XS, Pilot 200, Sion Blue, polymer jacket, or Runthrough wires. Fat from the thigh or leg was embolized and placed in the coronary blood vessel to mimic coronary plaque for the CTO procedure. A small incision adjacent to the right femoral artery access site was made and approximately 5 mm×10 mm pieces of fat were obtained. The fat was injected down the coronary guide into either the left circumflex or left anterior descending artery.

Each of the Ag/AgCl electrodes (hereafter W and C) were systematically placed at nine locations on the external skin surface of the subject, such that W and C maintained a certain axial distance for each test. The location of W and C, the current carrying electrodes, were selected to generate a current field that spanned the entire chest. Nine locations for W and C placement were selected from right internal jugular veins, left internal jugular vein, right lateral chest, right anterior chest, left anterior chest, left lateral chest, left femoral vein, left posterior chest, and right posterior chest. Four internal locations were also selected to act as W and C electrodes in some cases (FIG. 11A) including the right atrium, descending aorta, right coronary artery ostium and left main coronary artery ostium. The current carrying electrodes W and C were connected to the EIS device. A signal generator in the EIS device was used to supply a low amplitude AC current (10 μA to 1 mA, 1 kHz to 100 MHZ) to the current carrying electrodes W and C to generate an electric field.

Two intravascular guidewires with an exposed tip were used as the sensing electrodes (hereafter WS and R). Femoral 8Fr sheaths were placed to the right and left femoral arteries and the right femoral vein. A 7Fr JR 4 guide catheter was placed to the right coronary artery and a 7Fr AL 0.75 or JL4 was placed to the left coronary artery with dual transducers. The coronary guidewires were then placed down the coronaries through microcatheters. Other internal electrode positions were placed through a dual electrode pacing wire to the right atrium via the right femoral venous sheath, or via separate coronary wires through the sheaths to the aorta. The sensing electrodes WS and R were placed on the path of the current field (generated between W and C). The sensing electrode WS was positioned in any of the coronary vessels including the right coronary, left anterior descending, or left circumflex coronary artery. R was either a guidewire in blood (such as any coronary vessel, or the descending aorta or right atrium) or a skin electrode. To connect the guidewire electrodes to the EIS measurement device, a coaxial cable was clipped to the proximal/terminal exposed portion of each guidewire outside the body. In some implementations, a dual electrode pacer was positioned in the right atrium.

Standard iodine-based contrast was used for the study via a 3 port manifold allowing contrast injection, saline injection, and pressure monitoring for both the guidewires placed to the right and left coronaries. A standard ceiling mounted fluoroscopy system was used for the study as well.

When a low AC current was applied to W and C, a current field was generated in the blood stream, but also flows through other tissues which allows the measurements in non-blood tissues. The voltage difference across WS and R was measured. The voltage difference was used to calculate the apparent impedance magnitude, impedance trace, and the phase angle as shown in Equations 1-6. Different tissue types, such as blood, fat, myocardial space, subintimal space, and pericardial space in contact with WS and R were characterized using the impedance trace and magnitude. In some implementations, machine learning algorithms were used to classify the tissue type based on the impedance measurements.

About 173 tests were conducted both on a porcine and a cadaver for the various placements of the W and C and WS and R. The EIS system resulted in reliable Nyquist plot traces to distinguish the tissue types reliably, specifically at an input current value of 1 mA.

FIG. 11B illustrates an exemplary position of the 4-electrode system in a porcine. In this configuration, the current carrying electrodes W and C were positioned externally on the surface of the skin near the left internal jugular vein and left posterior chest. The sensing electrodes WS and R were positioned in the left circumflex, right coronary artery, or left descending artery. The real and imaginary components of apparent impedance were plotted on a Nyquist plot. The impedance trace for fat, blood, subintimal space, and myocardium were obtained as shown in FIG. 11B.

Data for characterizing tissue type during a coronary CTO intervention may be collected. The voltage measurements from the tests for CTO intervention may be used to calculate the apparent impedance magnitude, real and imaginary components of impedance, and the phase angle. A principal component analysis (PCA) algorithm may be used to categorize the data, distinguishing between blood, fat, myocardium space, and subintimal space. The PCA algorithm is a statistical technique used to reduce the dimensionality of data while preserving the most important information. The PCA algorithm helps to identify patterns in the EIS data by transforming high-dimensional impedance measurements (e.g., across multiple frequencies) into smaller set of uncorrelated features called as principal components. The PCA analysis demonstrated a reliable characterization of the various tissue types encountered during a CTO intervention. Other analyses methods may also be used in a similar manner to develop algorithms to classify tissue types in contact with the guidewire tip including machine learning methods (e.g., support vector machines, random forest, gradient boosting) or deep learning methods (e.g., convolutional neural networks, autoencoders).

In various implementations, a 2-, 3-, or 4-electrode system may be used to characterize tissue type during a CTO intervention. In some implementations, the current field is kept stable (W/C), and the voltage sensing electrode (WS/R) is mobile within the current field leading to varying geometrical configurations.

### E. Additional Examples

### 1. Example 1—Four-Electrode (Tetrapolar) Configuration for Real-Time Tissue Characterization

In some examples, techniques for four electrode (tetrapolar) configurations for real time tissue characterization are provided. The methods may include positioning one or a plurality of electrodes, configured as voltage sensing electrodes (working sense (WS)), adjacent to a tissue of interest within a subject. The WS electrode(s) may be affixed to an elongate member, such as a guidewire or a catheter. The positioning of the WS electrode(s) may include guiding a first electrode to contact the tissue of interest, and guiding a second electrode, configured as either an additional WS or as a reference (R) electrode, to a second location that is spatially separated from the first. In some instances, the second and any additional WS and/or R electrode(s) may be affixed to the same elongate member as the first WS electrode or to a separate and independent elongate member.

The methods may further involve positioning a plurality of current driving electrodes externally on the skin of the subject, or internally within the body. These current driving electrodes can be configured individually as either a counter electrode (C) or a working electrode (W) to form an anode cathode pair. This C and W pair is used to deliver an applied electrical signal adjacent to the tissue of interest. The C, W, and any additional C or W electrode(s) may be affixed to the same elongate members as the WS and R electrodes, to separate and individual elongate members, as external skin electrodes, or in combinations of the above.

In these methods, the applied electrical signal is a frequency sweep, defined as more than one distinct frequency step, which forms a minimum frequency band under test of at least 1 kHz. The applied signal is detected at the WS electrode(s) at each distinct frequency step, forming a recorded signal that is delivered to an external processor. The processor then calculates the resistive and reactive electrical spectra based on the voltage differential, phase differential, and the electrical field characteristics observed between the input signal and the recorded signal. Subsequently, one or more attributes associated with the calculated impedance spectra are evaluated.

Based on the one or more evaluated attributes, a tissue type of the tissue of interest immediately adjacent to at least one WS electrode is classified. The tissue type may be classified as, but is not limited to, plaque, blood, a subintimal layer, a pericardial space, fat, myocardium, muscle, or clot, among others. The resulting real time tissue classification is used by the operator for procedural guidance and tracking through endoluminal pathways and tissues that may otherwise be indistinguishable on common operating theatre imaging systems, such as fluoroscopy.

In some implementations of the methods, positioning the WS electrode adjacent to the tissue region of interest of the subject includes guiding the first WS electrode and a second WS or R electrode, or subsequent WS or R electrode(s), into the same blood vessel or cardiac chambers.

In other implementations, positioning the electrode adjacent to the tissue region of interest of the subject includes guiding the WS electrode and the second WS or R electrode, or subsequent WS or R electrode(s), into different blood vessels or cardiac chambers.

The step of positioning the electrode adjacent to the tissue region of interest may, in some configurations, involve guiding the first WS electrode and a second WS or R electrode, or subsequent WS or R electrode(s), into the same body cavity or lumen.

Alternatively, positioning the electrode adjacent to the tissue region of interest of the subject can include guiding the first WS electrode and the second WS or R electrode, or subsequent WS or R electrode(s), into different body cavities or lumens.

In some implementations, systems for performing the methods are provided. In these systems, the electrodes may be comprised of exposed, conductive surfaces on a commercially available guidewire. Alternatively, the first WS electrode, the second WS or R electrode, and any subsequent WS or R electrodes can be positioned axially on an elongate member, such as a guidewire. The WS or R electrodes may be positioned on a single, common elongate member and separated by an insulation material, or each WS or R electrode may be positioned on an individual member. In some configurations, the WS and R electrodes may be arranged in a combination of common and independent members. Furthermore, the WS electrode, the R electrode, and any additional WS or R electrode(s) are each individually connected to an electrical terminal at the proximal portion of the elongated member via an electrical conducting element.

Additionally, the elongate member, such as a guidewire or catheter, may be positioned within the central bore of a tubular member, for instance, one or more outer catheters. The tubular member can provide electrical insulation for the sensing electrode(s) from adjacent tissues when the electrode is positioned within the bore of the member. At any time throughout a procedure, the operator may modify the relative position of the tubular member with respect to the elongate member, which results in the sensing electrodes becoming either electrically coupled to the surrounding tissue or electrically isolated from it.

The methods may further include employing any of a WS or a R electrode as a W or C electrode. The configuration of the status of either electrode can be determined by software or through direct hardware configuration, for example, using a multiplexed relay, digital I/O from a processor, or a direct electrical connection made by the operator using conductive clips or similar means. In such configurations, an electrical signal is delivered to the C electrode to generate an applied electrical field. The transmission of this electrical field occurs via electrical conduction through or adjacent to the tissue of interest to a W electrode that is positioned internally within the subject or externally on the subject.

Additionally, positioning the plurality of electrodes, elongate members, guidewires, and catheters may include guiding each of these components through an artery or vein of the subject. This includes, but is not limited to, a right or left femoral artery or vein, a radial artery, a carotid artery, a subclavian artery, an axillary artery, a brachial artery or vein, or a jugular vein.

In other implementations, positioning the plurality of electrodes, elongate members, guidewires, and catheters includes guiding each of these components through any suitable blood vessel of the subject, accessible through percutaneous access or a surgical cut down.

In further implementations of the methods, positioning the plurality of electrodes, elongate members, guidewires, and catheters includes guiding each of these components through suitable lumens, which may be existing or surgically created.

The step of positioning the plurality of electrodes, elongate members, guidewires, and catheters may also include guiding each component through various body tissues. These tissues can include, but are not limited to, atherosclerotic plaque, vessel walls (subintima), myocardium, skeletal muscle, liver, subcutaneous tissue, or any other body tissue that might be intentionally or unintentionally accessed from blood vessels, blood containing cavities, or blood pools.

In some aspects, characterizing the tissue includes analyzing and comparing the applied versus recorded electrical signal data, including voltage amplitude, electrical impedance, electrical reactance, and phase shift, against a reference and/or a predetermined characterization dataset for each tissue type of interest. A processor executing impedance classification algorithms may be employed to make these comparisons of the electrical datasets. The reference itself may be acquired in real time or from previously stored datasets.

Characterizing the tissue can also include the visual inspection and real time analysis by an operator of a displayed trace or other visualization of the electrochemical impedance spectroscopy data.

The methods may further include determining an attribute associated with the location of the working sense electrode within a first blood vessel. This attribute can include identifying the location as being within blood, a subintimal layer, a pericardial space, the myocardium, or a plaque associated with that first vessel.

The electrical signal, in various implementations, may have a frequency range between 1 kilohertz (KHz) and 1 gigahertz (GHz). More specific frequency ranges can include 1 kHz to 500 megahertz (MHz), 1 kHz to 50 MHz, 1 kHz to 10 MHz, 1 kHz to 5 MHz, or 1 kHz to 1 MHz. The current amplitude may have a range between 0.1 to 500 microamperes (μA), or more specifically, 0.1 to 250 μA, 0.1 to 100 μA, or 1 to 100 μA.

According to the methods, applying the multi frequency electrical signal between the plurality of skin or internal current generating electrodes includes applying the signal in a manner that induces current flow through the subject's blood vessels and body tissue. The methods further include directing the positioning of the tissue of interest to be within the current field passing through that tissue.

In some configurations for positioning the internal electrodes, the methods include guiding a WS electrode into a coronary artery and guiding an R electrode into an arterial structure.

In other configurations, positioning the internal electrodes includes guiding a WS electrode into a coronary artery and guiding an R electrode into a cardiac vein, sinus, atrium, or ventricle.

Positioning the internal electrodes may also involve guiding a WS electrode into the coronary sinus or a cardiac vein structure, while guiding an R electrode into a cardiac venous or arterial structure.

In further implementations, positioning the internal electrodes can include guiding a WS electrode into the right atrium, right ventricle, pulmonary artery, pulmonary vein, left atrium, left ventricle, or aorta, and guiding an R electrode into a venous or arterial structure.

In specific applications of the methods, such as a peripheral intervention, positioning the internal electrodes includes guiding a WS electrode into a peripheral artery or vein, and guiding an R electrode into a peripheral artery or vein.

Positioning the internal electrodes may also involve guiding a WS electrode into a carotid artery, while an R electrode is guided into an arterial or venous structure.

In further configurations, positioning the internal electrodes includes guiding a WS electrode into a subclavian vein, jugular vein, the inferior vena cava, or the superior vena cava, and guiding an R electrode into an arterial or venous structure.

For neurovascular procedures, positioning the internal electrodes can include guiding a WS electrode into a cranial or cerebral artery or vein, and similarly guiding an R electrode into a cranial or cerebral artery or vein.

In other implementations, positioning the internal electrodes includes guiding a WS electrode into a hepatic artery or vein, and guiding an R electrode into a hepatic artery or vein, or another suitable arterial or venous structure.

Positioning the internal electrodes can also include guiding a WS electrode into a renal artery or vein, and guiding an R electrode into an arterial or venous structure, which may also be a renal artery or vein.

In general applications of the methods, positioning the internal electrodes includes guiding a WS electrode into any naturally occurring or surgically created lumen or cavity within the body, and guiding an R electrode into any naturally occurring or surgically created lumen or cavity within the body.

In some implementations, determining one or more attributes associated with the impedance spectra includes determining the impedance magnitude, phase, or spectral trace shape associated with the recorded impedance spectra. Classifying the tissue type adjacent to at least one WS electrode located at the tissue region of interest is then achieved by analyzing at least one of the impedance magnitude, the phase, or the spectral trace shape of the measured impedance spectra. This analysis involves applying one or more algorithmic approaches, including but not limited to dataset comparison, machine learning models, wavelet analysis, and artificial intelligence, to categorize the tissue type as one of, but not limited to, blood, fat, myocardial tissue, subintimal tissue, clot, muscle, or other tissue types.

### 2 Example 2—Three-Electrode (Tripolar) Configuration With Reconfigurable Sensing Electrode

In some examples, techniques for three electrode (tripolar) configurations with a reconfigurable sensing electrode are provided. The methods for characterizing tissue in real time during coronary intervention procedures may include positioning a minimum of three electrodes, configured as any combination of W+WS+R/C, W+WS/R+C, or W+WSvR+C electrodes. In many cases, the WS electrode is positioned adjacent to a tissue of interest within a subject. The WS electrode, in any paired or unpaired combination, is affixed to an elongate member such as a guidewire or a catheter, and its positioning includes at least guiding the electrode to be adjacent to a tissue of interest. A key aspect of these methods is that the WS electrode may be configurable as an R electrode at different time points during a procedure for the purpose of obtaining an electrical reference baseline measurement. The WS, R, and any additional WS or R electrode(s) may be affixed to the same elongate members or to separate and individual elongate members or combinations of the two.

The methods further involve positioning a plurality of current driving electrodes externally on the skin of the subject or internally within the body. These electrodes may be configured individually as either a counter electrode (C) or a working electrode (W), forming an anode cathode pair used to deliver an applied electrical signal adjacent to the tissue of interest. The C, W, and any additional C or W electrode(s) may be affixed to the same elongate members as the WS and R electrodes, to separate and individual elongate members, as external skin electrodes, or in combinations thereof. The applied electrical signal is a frequency sweep, and this signal is detected at the WS electrode(s), forming a recorded signal that is delivered to an external processor. The processor then calculates the resistive and reactive electrical spectra, evaluates one or more attributes associated with the spectra, and classifies the adjacent tissue type as, but not limited to, plaque, blood, a subintimal layer, a pericardial space, fat, myocardium, muscle, or clot. The resulting real time tissue classification is used by the operator for procedural guidance and tracking.

In specific applications of the methods, such as a peripheral intervention, positioning the internal electrodes includes guiding a WS electrode into a peripheral artery or vein, and guiding an R electrode into a peripheral artery or vein.

Positioning the internal electrodes may also involve guiding a WS electrode into a carotid artery, while an R electrode is guided into an arterial or venous structure.

In further configurations, positioning the internal electrodes includes guiding a WS electrode into a subclavian vein, jugular vein, the inferior vena cava, or the superior vena cava, and guiding an R electrode into an arterial or venous structure.

For neurovascular procedures, positioning the internal electrodes can include guiding a WS electrode into a cranial or cerebral artery or vein, and similarly guiding an R electrode into a cranial or cerebral artery or vein.

In other implementations, positioning the internal electrodes includes guiding a WS electrode into a hepatic artery or vein, and guiding an R electrode into a hepatic artery or vein, or another suitable arterial or venous structure.

Positioning the internal electrodes can also include guiding a WS electrode into a renal artery or vein, and guiding an R electrode into an arterial or venous structure, which may also be a renal artery or vein.

In general applications of the methods, positioning the internal electrodes includes guiding a WS electrode into any naturally occurring or surgically created lumen or cavity within the body, and guiding an R electrode into any naturally occurring or surgically created lumen or cavity within the body.

In some implementations, determining one or more attributes associated with the impedance spectra includes determining the impedance magnitude, phase, or spectral trace shape associated with the recorded impedance spectra. Classifying the tissue type adjacent to at least one WS electrode located at the tissue region of interest is then achieved by analyzing at least one of the impedance magnitude, the phase, or the spectral trace shape of the measured impedance spectra. This analysis involves applying one or more algorithmic approaches, including but not limited to dataset comparison, machine learning models, wavelet analysis, and artificial intelligence, to categorize the tissue type as one of, but not limited to, blood, fat, myocardial tissue, subintimal tissue, clot, muscle, or other tissue types.

In further implementations of the methods, positioning the WS electrode adjacent to the tissue region of interest of the subject includes guiding the WS, WS/R, or WSVR electrode and an additional or subsequent, separate WS or R electrode(s) into the same blood vessel.

Alternatively, positioning the electrode adjacent to the tissue region of interest can include guiding the WS, WS/R, or WSVR electrode and an additional or subsequent, separate WS or R electrode(s) into different blood vessels.

In other configurations of the methods, positioning the electrode adjacent to the tissue region of interest of the subject includes guiding the first WS, WS/R, or WSVR electrode and an additional or subsequent, separate WS or R electrode(s) into the same body cavity or lumen.

Positioning the electrode adjacent to the tissue region of interest may also involve guiding the first WS, WS/R, or WSVR electrode and an additional or subsequent, separate WS or R electrode(s) into different body cavities or lumens.

In some implementations, the first WS electrode, the R electrode, and any subsequent WS or R electrodes are positioned axially on an elongate member, such as a guidewire. The WS or R electrodes may be positioned on a single, common elongate member and separated by an insulation material; they may be one and the same electrode or each may be a distinctly separate electrode. Alternatively, each WS or R electrode may be positioned on an individual elongate member, or the WS and R electrodes may be configured in any combination of common and independent elongate members. The WS electrode, the R electrode, and any additional WS or R electrode(s) are connected to an electrical terminal at the proximal portion of the elongated member via an electrical conducting element.

The methods may also be performed such that the elongate member, such as a guidewire or catheter, is positioned within the central bore of a tubular member, for example, an outer catheter. In such cases, the tubular member may provide electrical insulation for the sensing electrode(s) from the adjacent tissues when the electrode is positioned within the bore. The operator may modify the relative position of the tubular member to the elongate member at any time throughout a procedure, resulting in the sensing electrodes becoming either electrically coupled to surrounding tissue or electrically isolated from it.

The methods may further include employing any of a WS or a R electrode as a W or C electrode. The configuration of the status of either electrode can be determined by software or by direct hardware configuration. An electrical signal is delivered to the C electrode to generate an applied electrical field, and the transmission of this field occurs via electrical conduction through or adjacent to the tissue of interest to a W electrode positioned internally within the subject or externally on the subject.

In specific implementations, positioning the plurality of electrodes, elongate members, guidewires, and catheters includes guiding each of these components through a right femoral artery, a left femoral artery, a right femoral vein, or a left femoral vein of the subject.

In other implementations, positioning the plurality of electrodes, elongate members, guidewires, and catheters includes guiding each of these components through suitable blood vessels of the subject, which are accessible through percutaneous access or a surgical cut down.

Furthermore, positioning the plurality of electrodes, elongate members, guidewires, and catheters can include guiding each of these components through suitable lumens, whether they are existing or surgically created.

In some implementations, characterizing the tissue includes analyzing and comparing the applied versus recorded electrical signal data, including voltage amplitude, electrical impedance, electrical reactance, and phase shift, against an electrical reference value and a predetermined characterization dataset for each tissue type of interest. A processor executing impedance classification algorithms is employed to make these comparisons of the electrical datasets. The reference may be acquired in real time or from stored datasets. The electrical reference may be one of several types. For example, it can be a virtual reference, recorded using a WS/R or WSVR electrode configured in R mode to record a reference electrical spectrum from a point close to, but not directly adjacent to, the tissue of interest, such as from blood. In this case, the operator physically guides the electrode to a suitable reference point where a spectrum is captured and stored on a processor, before guiding the electrode to the tissues of interest for characterization in the WS configuration. Alternatively, the electrical reference may be taken from an R/C electrode, operating in an R configuration, to obtain a reference electrical spectrum at a point spatially separate from the WS electrode. In another approach, an electrical reference may be derived from a cohort of subjects, using an R electrode to obtain an average population estimate of the reference electrical spectrum for that tissue type in that body location.

The methods of characterizing the tissue may also include the visual inspection and real time analysis by an operator of a displayed trace or other visualization of the electrochemical impedance spectroscopy data.

The methods can further include determining an attribute associated with the location of the WS electrode within a first blood vessel. This attribute may include identifying the location as being in blood, a subintimal layer, a pericardial space, the myocardium, or an atherosclerotic plaque, thrombus, or another tissue type associated with the first vessel.

In various disclosed implementations, the electrical signal has a frequency range between 1 kilohertz (kHz) and 1 gigahertz (GHz). Other possible ranges include 1 kHz to 500 megahertz (MHZ), 1 kHz to 50 megahertz (MHz), 1 kHz to 10 megahertz (MHZ), 1 kHz to 5 megahertz (MHZ), or 1 kHz to 1 megahertz (MHz). The current amplitude may have a range between 0.1 to 500 microamperes (μA), or more specifically, 0.1 to 250 μA, 0.1 to 100 μA, or 1 to 100 ρA.

In a further aspect of the methods, applying the multi frequency electrical signal between the plurality of skin or internal current generating electrodes includes applying the signal to induce current flow through the subject's blood vessels and body tissue. The methods further include directing the current field through the tissue of interest.

In some configurations, positioning the internal electrodes may include guiding a WS, WS/R, R/C, or R electrode into a coronary artery.

Positioning the internal electrodes may also include guiding a WS, WS/R, R/C, or R electrode into a cardiac vein, sinus, atrium, or ventricle.

The step of positioning the internal electrodes can also involve guiding a WS, WS/R, R/C, or R electrode into a coronary venous or arterial structure.

In other implementations, positioning the internal electrodes includes guiding a WS, WS/R, R/C, or R electrode into a peripheral artery or vein.

For neurovascular applications, positioning the internal electrodes may include guiding a WS, WS/R, R/C, or R electrode into a cranial or cerebral artery or vein.

In other applications of the methods, positioning the internal electrodes includes guiding a WS, WS/R, R/C, or R electrode into a hepatic artery or vein.

The step of positioning the internal electrodes can also involve guiding a WS, WS/R, R/C, or R electrode into a renal artery or vein.

In more general implementations, positioning the internal electrodes includes guiding a WS, WS/R, R/C, or R electrode into any naturally occurring or surgically created lumen or cavity within the body.

In a further aspect of the methods, determining one or more attributes associated with the impedance spectra includes determining the impedance magnitude, phase, or spectral trace shape associated with the recorded impedance spectra. Subsequently, the tissue type adjacent to at least one WS electrode located at the tissue region of interest is classified by analyzing at least one of the impedance magnitude, the phase, or the spectral trace shape associated with the measured impedance spectra. This classification also involves applying one or more algorithmic approaches, including but not limited to dataset comparison, machine learning models, and wavelet analysis.

### 3. Example 3—Two-Electrode (Bipolar) Configuration for Real-Time Tissue Characterization

In some examples, techniques for two electrode (bipolar) configurations for real time tissue characterization are provided. The methods include positioning a minimum of two electrodes, configured as a reference counter (R/C) electrode and a working sense (W/WS) electrode, adjacent to a tissue of interest within a subject. In these configurations, the W/WS electrode is affixed to an elongate member such as a guidewire or a catheter. The positioning of the electrodes includes at least guiding a first W/WS electrode adjacent to the tissue of interest, and guiding the R/C electrode to a second location that is spatially separated from the first. The R/C electrode and any additional counter, working sense, or reference electrode(s) may be affixed to the same elongate members, to separate and individual elongate members, or in combinations of the two.

According to these methods, an electrical signal is generated between the R/C and W/WS electrodes adjacent to the tissue of interest. The applied electrical signal is a frequency sweep, and this signal is detected at the W/WS electrode(s), forming a recorded signal that is delivered to an external processor. The processor then calculates the resistive and reactive electrical spectra based on the voltage differential, phase differential, and the electrical field characteristics observed between the input and recorded signals.

Following the calculation, one or more attributes associated with the calculated impedance spectra are evaluated. Based on these attributes, a tissue type present immediately adjacent to at least one W/WS electrode is classified as one of, but not limited to, plaque, blood, a subintimal layer, a pericardial space, fat, myocardium, muscle, or clot, among others. The resulting real time tissue classification is used by the operator for procedural guidance and tracking through endoluminal pathways and tissues that may otherwise be indistinguishable on common operating theatre imaging systems like fluoroscopy.

In further implementations of the methods, positioning the electrode adjacent to the tissue region of interest of the subject includes guiding both the W/WS and the R/C electrode into the same tissue of interest, for example, plaque, adipose tissue, or myocardium.

In other configurations, positioning the electrode adjacent to the tissue region of interest involves guiding the W/WS and the R/C electrode into the same blood vessel.

The step of positioning the electrode adjacent to the tissue region of interest may also include guiding a W/WS electrode and an additional or subsequent, separate R/C electrode into different blood vessels.

According to other aspects of the methods, positioning the electrode adjacent to the tissue region of interest includes guiding the first W/WS electrode and the R/C electrode into the same body cavity or lumen.

Alternatively, positioning the electrode adjacent to the tissue region of interest of the subject may include guiding the W/WS electrode and an R/C electrode into different body cavities or lumens.

In some implementations, the W/WS electrode and the R/C electrode are positioned axially on an elongate member, such as a guidewire. These electrodes may be positioned on a single, common elongate member and separated by an insulation material. Alternatively, each of the W/WS and R/C electrodes may be positioned on an individual elongate member, or they may be configured in any combination of common and independent elongate members. The W/WS electrode, the R/C electrode, and any additional W/WS or R/C electrode(s) are connected to an electrical terminal at the proximal portion of the elongated member via an electrical conducting element.

The methods may also be performed such that the elongate member, such as a guidewire or catheter, is positioned within the central bore of a tubular member like an outer catheter. The tubular member can provide electrical insulation for the electrode(s) from adjacent tissues when positioned within its bore. The operator may modify the relative position of the tubular member to the elongate member at any time during a procedure, which can result in the electrodes becoming electrically coupled to or isolated from the surrounding tissue.

In these methods, the W/WS electrode can act as a current injecting electrode and a voltage sense electrode simultaneously, while the R/C electrode can act as a current sink or a voltage sense electrode simultaneously. An electrical signal is delivered to the R/C electrode to generate an applied electrical field. The transmission of this electrical field occurs via electrical conduction through or adjacent to the tissue of interest to the W/WS electrode, which is positioned internally within the subject or externally on the subject.

In certain procedures, positioning the plurality of electrodes, elongate members, guidewires, and catheters includes guiding each of these components through a right femoral artery, a left femoral artery, a right femoral vein, or a left femoral vein of the subject.

More broadly, positioning the plurality of electrodes, elongate members, guidewires, and catheters can include guiding each component through any suitable blood vessels of the subject that are accessible through percutaneous access or a surgical cut down.

The step of positioning the plurality of electrodes, elongate members, guidewires, and catheters may also include guiding each of these components through suitable lumens, whether they are existing or have been surgically created.

In further aspects of the methods, characterizing the tissue includes analyzing and comparing the applied versus recorded electrical signal data (i.e., the voltage phasor) against an electrical reference value and a predetermined characterization dataset for each tissue type of interest. To facilitate this, a processor executing impedance classification algorithms is employed to make comparisons of the electrical datasets. The reference itself may be acquired in real time or from stored datasets. The electrical reference can be one of several types. For instance, it may be a virtual reference, recorded using the R/C electrode to obtain a reference electrical spectrum from a point close to, but not directly adjacent to, the tissue of interest, such as from blood. In this case, the operator physically guides the R/C electrode to a suitable reference point to capture and store the reference spectrum before guiding the W/WS electrode to the tissues of interest for characterization. Alternatively, the electrical reference may be taken from an R/C electrode at a point spatially separate from the W/WS electrode. In another approach, an electrical reference may be derived from a cohort of subjects, using an R electrode to obtain an average population estimate of the reference electrical spectrum for that tissue type in that specific body location.

Characterizing the tissue may also include the visual inspection and real time analysis of a displayed trace or other visualization of the electrochemical impedance spectroscopy data. This visualization can be provided via a custom graphic user interface or by using common plots, such as a Nyquist, Cole-Cole, or Bode plot.

The methods can further include determining an attribute associated with the location of the W/WS electrode within a first blood vessel. This attribute includes identifying the location as being within blood, a subintimal layer, a pericardial space, the myocardium, or an atherosclerotic plaque, thrombus, or another tissue type associated with the vessel.

In various disclosed implementations, the applied electrical signal has a frequency range between 1 kilohertz (kHz) and 1 gigahertz (GHz). More specific frequency ranges can include 1 kHz to 500 megahertz (MHz), 1 kHz to 50 MHz, 1 kHz to 10 MHZ, 1 kHz to 5 MHz, or 1 kHz to 1 MHz. Correspondingly, the current amplitude may have a range between 0.1 to 500 microamperes (μA), or more specifically, 0.1 to 250 μA, 0.1 to 100 μA, or 1 to 100 μA.

The step of applying the multi frequency electrical signal between the plurality of skin or internal current generating electrodes includes applying the signal to induce current flow through the subject's blood vessels and body tissue. The methods further include directing the resulting current field through the tissue of interest.

In some implementations, positioning the internal electrodes may include guiding a W/WS or R/C electrode into a coronary artery.

Positioning the internal electrodes may also include guiding a W/WS or R/C electrode into a cardiac vein, sinus, atrium, or ventricle.

The methods may also involve positioning the internal electrodes by guiding a W/WS or R/C electrode into a coronary venous or arterial structure.

For peripheral applications, positioning the internal electrodes may include guiding a W/WS or R/C electrode into a peripheral artery or vein.

In neurovascular procedures, positioning the internal electrodes can include guiding a W/WS or R/C electrode into a cranial or cerebral artery or vein.

In other applications of the methods, positioning the internal electrodes includes guiding a W/WS or R/C electrode into a hepatic artery or vein.

The step of positioning the internal electrodes may also involve guiding a W/WS or R/C electrode into a renal artery or vein.

In general implementations, positioning the internal electrodes includes guiding a W/WS or R/C electrode into any naturally occurring or surgically created lumen or cavity within the body.

In further aspects of the methods, determining one or more attributes associated with the impedance spectra includes determining the impedance magnitude, phase, or spectral trace shape associated with the recorded impedance spectra. The tissue type adjacent to at least one WS electrode that is located at the tissue region of interest is then classified by analyzing at least one of the impedance magnitude, the phase, or the spectral trace shape of the measured impedance spectra, and by applying one or more algorithmic approaches including but not limited to dataset comparison, machine learning models, and wavelet analysis.

While this specification contains many specific implementation details, these should not be construed as limitations on the scope of claimed subject matter, but rather as descriptions of features specific to particular implementations. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method for characterizing biological tissue for an endoluminal procedure, the method comprising:
positioning a first electrode within an endoluminal space adjacent to a region of biological tissue;
positioning a second electrode spatially separated from the first electrode;
applying a variable-frequency input electrical signal across the first electrode and the second electrode to generate an electrical field corresponding to the region;
measuring a voltage response between the first electrode and the second electrode based on the electric field;
determining a voltage phasor based on the voltage response relative to the variable-frequency input electrical signal; and
determining a characteristic of the region based on the voltage phasor.

2. The method of claim 1, further comprising:
positioning a third electrode and a fourth electrode that are each spatially separated from the first electrode;
wherein the variable-frequency input electrical signal is applied across the third electrode and the fourth electrode; and
wherein the voltage response is measured between the first electrode and the second electrode.

3. The method of claim 1, wherein:
the first electrode is carried by an elongated member; and
the elongated member comprises a guidewire, a catheter, or a sheath.

4. The method of claim 3, wherein:
the elongated member comprises an insulating sheath; and
the method further comprises retracting the insulating sheath relative to a body of the elongated member to adjust a length of an exposed region of the first electrode.

5. The method of claim 1, wherein the region of biological tissue comprises a body vessel.

6. The method of claim 1, wherein positioning the second electrode comprises positioning the second electrode on an external skin surface of a subject.

7. The method of claim 1, wherein the variable-frequency input electrical signal comprises a frequency sweep across a test frequency band.

8. The method of claim 7, wherein:
the test frequency band is within a range of 1 kilohertz to 1 gigahertz; and
the input electrical signal has a current amplitude within a range of 0.1 microamperes to 10 milliamperes.

9. The method of claim 1, wherein determining the characteristic of the region comprises comparing the determined voltage phasor to a reference dataset using a machine learning model.

10. The method of claim 1, further comprising:
displaying, on a user interface, a graphical representation of the characteristic.

11. The method of claim 1, wherein the biological tissue comprises cardiovascular, neurological, hepatic, renal, or musculoskeletal structures.

12. The method of claim 1, wherein the characteristic identifies the region of biological tissue as one or more materials selected from a group comprising blood, subintimal layer, vessel wall, plaque, myocardium, pericardial space, fat, muscle, and clot.

13. The method of claim 1, further comprising:
navigating the first electrode through a vascular pathway based on the characteristic.

14. A system for characterizing biological tissue for an endoluminal procedure, the system comprising:
an elongated member having a first electrode, wherein the first electrode is configured for positioning within an endoluminal space adjacent to a region of the biological tissue;
a second electrode configured for positioning to be spatially separated from the first electrode;
a signal generator operatively coupled to each of the first electrode and the second electrode, wherein the signal generator is configured to provide a variable-frequency input electrical signal across the first electrode and the second electrode to generate an electric field through the region;
a measurement unit operatively coupled to each of the first electrode and the second electrode, wherein the measurement unit is configured to measure a voltage response between the first electrode and the second electrode based on the electric field; and
a processor operatively coupled to each of the measurement unit and the signal generator, wherein the processor is configured to:
receive data indicating the voltage response;
determine a voltage phasor based on the voltage response relative to the variable-frequency input electrical signal; and
generate output data comprising a characteristic of the region based on the voltage phasor.

15. The system of claim 14, wherein:
the elongated member includes a proximal electrical interface; and
the first electrode is electrically coupled to the proximal electrical interface via a conductor disposed in the elongated member.

16. The system of claim 14, further comprising:
a user interface configured to receive the output data from the processor and provide a visual representation of the characteristic.

17. The system of claim 14, wherein the first electrode comprises a conductive surface on the elongated member that is electrically isolated from a body of the elongated member by an insulating material.

18. The system of claim 14, wherein the processor is further configured to generate a control signal that, when received by the signal generator, causes the signal generator to adjust one or more of an excitation frequency or a signal amplitude of the variable-frequency input electrical signal based on the voltage response.

19. The system of claim 14, wherein the processor is configured to generate the output data by executing a machine learning model to compare the voltage phasor to a reference dataset.

20. The system of claim 14, further comprising:
a third electrode;
a fourth electrode; and
wherein the signal generator is operatively coupled to the third electrode and the fourth electrode and configured to apply the variable-frequency input electric signal across the third electrode and the fourth electrode.
